(19) United States
(12) Patent Application Publication
(10) Pub. No.: US 2004/0018968 A1
(43) Pub. Date: Jan. 29, 2004

(54) Use of histone deacetylase inhibitors in combination with radiation for the treatment of cancer

(76) Inventors: George Sgouros, Ellicott City, MD (US); Victoria M. Richon, Rye, NY (US); Paul A. Marks, Washington, CT (US); Richard A. Rifkind, New York, NY (US)

Correspondence Address: MINTZ, LEVIN, COHN, FERRIS, GLOVSKY & POPEO,P.C., ONE FINANCIAL CENTER, BOSTON, MA, 02111, US

(21) Appl. No.: 10/413,422

(22) Filed: Apr. 15, 2003

Related U.S. Application Data
(60) Provisional application No. 60/373,033, filed on Apr. 15, 2002.

(51) Int. Cl. A61K 38/12; A61N 5/00; A61K 31/19

(52) U.S. Cl. 514/9; 514/557; 514/575; 600/1

(57) ABSTRACT

The present invention relates to a method for the treatment of cancer in a patient in need thereof. The method comprises administering to a patient in need thereof a first amount of a histone deacetylase inhibitor in a first treatment procedure, and a second amount or dose of radiation in a second treatment procedure. The first and second treatments together comprise a therapeutically effective amount. The combination of the HDAC inhibitor and radiation therapy is therapeutically synergistic.

### RELATED APPLICATIONS

This application claims the benefit of U.S. Provisional Application No. 60/373,033 filed on Apr. 15, 2002. The entire teachings of the above-referenced application are incorporated herein by reference.

### GOVERNMENT SUPPORT

The invention was supported, in whole or in part, by a Core Grant (Grant No. 08748) from the National Cancer Institute and CA 05826 from NIH. The Government has certain rights in the invention.

### BACKGROUND OF THE INVENTION

Normal tissue homeostasis is achieved by an intricate balance between the rate of cell proliferation and cell death. Disruption of this balance either by increasing the rate of cell proliferation or decreasing the rate of cell death can result in the abnormal growth of cells and is thought to be a major event in the development of cancer. Conventional strategies for the treatment of cancer include chemotherapy, radiotherapy, surgery, biological therapy or combinations thereof; however these strategies are limited by lack of specificity and excessive toxicity to normal tissues. In addition, certain cancers are refractory to treatments such as chemotherapy, and some of these strategies such as surgery are not always viable alternatives.

Cancer cells can be weakened and ultimately killed by bombardment with certain kinds of radiation, and thus radiation therapy is an important treatment for cancer. Retrospective analyses of cancer radiotherapy, for example in the case of prostate cancer, have demonstrated that failure to achieve local control of the primary tumor is strongly associated with eventual metastatic dissemination of disease (Yorke, E. D. et al. Cancer Res. 53: 2987-93(1993); Fuks, Z. et al. Int. J. Radiat. Onco.l Biol. Phys. 21. 537-47(1991)). The availability of early markers of recurrence, such as PSA, have also suggested that the standard dosing regimens used in radiotherapy of prostate cancer are inadequate (Pollack, A. et al. Int J Radiat Oncol Biol Phys. 53: 1097-1105 (2002)). These two observations have provided an impetus for the investigation of techniques such as 3-D conformal treatment and intensity modulated radiotherapy (IMRT) that make it possible to increase the therapeutic radiation dose with minimal increases in normal organ exposure (Zelefsky, M. J. et al. Radiother. Oncol. 55: 241-9(2000)). The use of radiosensitizers as an approach to increase therapeutic efficacy without increasing dose delivery has also been examined (Lawton, C. A. et al. Int. J. Radiat. Oncol. Biol. Phys. 36: 673-80 (1996)).

Cancer treatment can also include the use of chemotherapeutic agents. For example, Suberoylanilide Hydroxamic Acid (SAHA) is a hydroxamic acid-based hybrid polar compound that inhibits histone deacetylase (HDAC) activity and that induces terminal differentiation, cell growth arrest and/or apoptosis of tumor cells, in vitro (Richon, V. M. et al. Proc. Natl. Aca.d Sci. USA. 95: 3003-7 (1998); Marks, P. A. et al. Curr. Opin. Oncol. 13: 477-83 (2001); Marks, P. A. et al. Nature Reviews Cancer 1: 194-202 (2001)). SAHA belongs to a class of histone deacetylase (HDAC) inhibitors capable of inducing terminal differentiation, cell growth arrest and/or apoptosis of tumor cells. The compound has shown inhibition of prostate tumor xenografts in nude mice with minimal to no detectable toxicity (Butler, L. M. et al. Cancer Res. 60: 5165-70 (2000). It has completed Phase I trials for the treatment of solid and hematological tumors, including prostate cancer (Kelly, W. K. et al. Expert Opin. Investig. Drugs 11: 1695-713 (2002); Kelly, W. K. et al. In: ASCO Proceedings, Orlando, Fla., 2002, pp. 1831).

Typically, HDAC inhibitors fall into five general classes: A) Hydroxamic acid derivatives; B) Cyclic tetrapeptides; C) Short Chain Fatty Acids (SCFAs); D) Benzamide derivatives; and E) Electrophilic ketone derivatives.

Combination therapies are often employed in cancer treatment. For example, two or more accepted therapies, such as, chemotherapy and radiotherapy have been employed. The therapeutic gain derived from certain combination therapies has been classified under four general categories by Steel and Peckham (Int. J. Radiat. Oncol. Biol. Phys. 5: 85-91(1979)). These categories are: 1) Spatial Cooperation—chemotherapy and radiotherapy eradicate disease in different anatomical sites; 2) Toxicity Independence—kill due to chemotherapy is added to that derived from radiotherapy because of non-overlapping normal organ toxicity; 3) Normal Tissue Protection—agents that make it possible to deliver larger doses of radiation to the target; 4) Enhancement of Tumor Response—one agent (chemotherapy or radiation) preferentially “sensitizes” tumor cells to the other such that the effect of the two is greater than would be expected by adding the effect of each individually.

The first two categories do not require an interaction between the two agents. Clinical examples of therapeutic gain due to combined radiotherapy/chemotherapy generally fall under categories 1 and 2, with category 1 being the dominant clinical rationale for combined modality therapy. Therapeutic gains corresponding to categories 3 and 4 have been observed in the laboratory but translation to the clinic has been slow.

In view of the above, cancer is a disease for which many potentially effective treatments are available. However, due to the prevalence of cancers of various types and the serious effects it can have, more effective treatments, especially those with fewer adverse side effects than currently available forms of treatment, are needed.

### SUMMARY OF THE INVENTION

The present invention is based on the discovery that histone deacetylase (HDAC) inhibitors, such as SAHA can be used in combination with a radiation source such as external beam irradiation or a radioisotope, such as a radiopharmaceutical, to provide therapeutically effective anticancer effects. Furthermore, an unexpected synergistic interaction between the HDAC inhibitor and the radiation source results in an enhanced or synergistic therapeutic effect, wherein the combined effect is greater than the additive effect resulting from administration of the two treatments each at a therapeutic dose. These observations suggest that HDAC inhibitors, such as SAHA, can act as radiosensitizers that can be used in combination with radiotherapy for the treatment of cancer. The ability of HDAC inhibitors such as SAHA to act as radiosensitizers has not been previously described.

It has been unexpectedly discovered that the combination of a first treatment procedure which includes administration of a histone deacetylase (HDAC) inhibitor, as described herein, and a second treatment procedure using radiation treatment, as described herein, to a patient in need thereof can provide therapeutically effective anticancer effects. Each of the treatments (administration of an HDAC inhibitor and administration of radiation therapy) is used in an amount or dose which in combination with the other provides a therapeutically effective treatment.

As such, the present invention relates to a method for the treatment of cancer in a patient in need thereof. Treatment of cancer, as used herein, refers to partially or totally inhibiting, delaying or preventing the progression of cancer including cancer metastasis; inhibiting, delaying or preventing the recurrence of cancer including cancer metastasis; or preventing the onset or development of cancer (chemoprevention) in a mammal, for example a human.

The methods of the present invention are useful in the treatment of a wide variety of cancers, including but not limited to solid tumors (e.g., tumors of the lung, breast, colon, prostate, bladder, rectum, brain or endometrium), hematological malignancies (e.g., leukemias, lymphomas, myelomas), carcinomas (e.g. bladder carcinoma, renal carcinoma, breast carcinoma, colorectal carcinoma), neuroblastoma, or melanoma.

The method comprises administering to a patient in need thereof a first amount of a histone deacetylase inhibitor in a first treatment procedure, and a second amount or dose of radiation in a second treatment procedure. The first and second amounts together comprise a therapeutically effective amount.

The invention further relates to pharmaceutical composition useful for the treatment of cancer. The pharmaceutical composition comprises a first amount of a histone deacetylase inhibitor and a second amount of radiation (e.g., a radiopharmaceutical). The first and second amount together comprise a therapeutically effective amount.

The invention further relates to the use of a first amount of an HDAC inhibitor and a second amount of a radiation (e.g., a radiopharmaceutical agent) for the manufacture of a medicament for treating cancer.

In particular embodiments of this invention, the combination of the HDAC inhibitor and radiation therapy is considered therapeutically synergistic when the combination treatment regimen produces a significantly better anticancer result (e.g., inhibition of growth) than the additive effects of each constituent when it is administered alone at a therapeutic dose. Standard statistical analysis can be employed to determine when the results are significantly better. For example, a Mann-Whitney Test or some other generally accepted statistical analysis can be employed.

The radiation source used in the radiation treatment can be electromagnetic radiation (e.g. X-ray or gamma rays), or particulate radiation (e.g. electron beams (beta particles), protons beams, neutron beams, alpha particles, or negative pi mesons).

The radiation treatment can be external beam radiation, or can involve the use of a radioisotope (e.g., by administration of a radiopharmaceutical agent, as described herein). The radiation treatment can also be a combination of external beam radiation and a radioisotope, such as a radiopharmaceutical agent.

In one particular embodiment, the radiation is provided by targeted delivery or by systemic delivery of targeted radioactive conjugates, for example a radiolabeled antibody.

The dose of radiation can be determined depending on the patient, and the type of cancer being treated. In particular embodiments, the patient can receive at least about 1 Gy of radiation, for example about 5-40 Gy of radiation such as about 5, 6, 7, 8, 9 or 10 Gy, 20 Gy or 40 Gy of radiation and the like.

The treatment procedures can take place sequentially in any order, simultaneously or a combination thereof. For example, the first treatment procedure, administration of a histone deacetylase inhibitor, can take place prior to the second treatment procedure, radiation, after the radiation treatment, at the same time as the radiation or a combination thereof. For example, a total treatment period can be decided for the histone deacetylase inhibitor. The radiation can be administered prior to onset of treatment with the inhibitor or following treatment with the inhibitor. In addition, radiation treatment can be administered during the period of inhibitor administration but does not need to occur over the entire inhibitor treatment period.

HDAC inhibitors suitable for use in the present invention, include but are not limited to hydroxamic acid derivatives, Short Chain Fatty Acids (SCFAs), cyclic tetrapeptides, benzamide derivatives, or electrophilic ketone derivatives, as defined herein.

Specific non-limiting examples of HDAC inhibitors suitable for use in the methods of the present invention are:

A) HYDROXAMIC ACID DERIVATIVES selected from SAHA, pyroxamide, CBHA, Trichostatin A (TSA), Trichostatin C, Salicylihydroxamic Acid (SBHA), Azelaic Bishydroxamic Acid (ABHA), Azelaic-1-Hydroxamate-9-Anilide (AAHA), 6-(3-Chlorophenylureido) carpoic Hydroxamic Acid (3Cl-UCHA), Oxamflatin, A-161906, Scriptaid, PXD-101, LAQ-824, CHAP, MW2796, and MW2996;

B) CYCLIC TETRAPEPTIDES selected from, Trapoxin A, FR901228 (FK 228, Depsipeptide), FR225497, Apicidin, CHAP, HC-Toxin, WF27082, and Chlamydocin;

C) SHORT CHAIN FATTY ACIDS (SCFAs) selected from Sodium Butyrate, Isovalerate, Valerate, 4 Phenylbutyrate (4-PBA), Phenylbutyrate (PB), Propionate, Butyramide, Isobutyramide, Phenylacetate, 3-Bromopropionate, Tributyrin, Valproic acid and Valproate;

D) BENZAMIDE DERIVATIVES selected from CI-994, MS-27-275 (MS-275) and a 3′-amino derivative of MS-27-275;

E) ELECTROPHILIC KETONE DERIVATIVES selected from a trifluoromethyl ketone and an α-keto amide such as an N-methyl-α-ketoamide; and

F) DEPUDECIN.

Specific HDAC inhibitors include:

Suberoylanilide hydroxamic acid (SAHA), which is represented by the following structural formula:

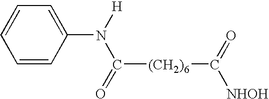

Pyroxamide which is represented by the following structural formula:

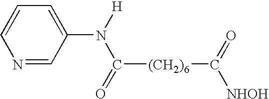

m-carboxycinnamic acid bishydroxamate (CBHA) which is represented by the structural formula:

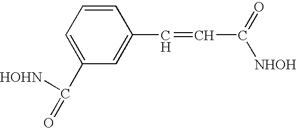

Other non-limiting examples of HDAC inhibitors which are suitable for use in the methods of the present invention are:

A compound represented by the structure:

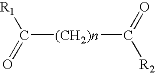

wherein R₁ and R₂ can be the same or different; when R₁ and R₂ are the same, each is a substituted or unsubstituted arylamino (e.g., phenylamino, pyridineamino, 9-purine-6-amino or thiazoleamino), cycloalkylamino, or piperidino group; when R₁ and R₂ are different R₁═R₃—N—R₄, wherein each of R₃ and R₄ are independently the same as or different from each other and are a hydrogen atom, a hydroxyl group, a substituted or unsubstituted, branched or unbranched alkyl, alkenyl, cycloalkyl, aryl (e.g., phenyl or pyridyl), alkyloxy, aryloxy, arylalkyloxy or pyridine group, or R₃ and R₄ are bonded together to form a piperidine group, R₂ is a hydroxylamino, hydroxyl, amino, alkylamino, dialkylamino or alkyloxy group and n is an integer from about 4 to about 8;

A compound represented by the structure:

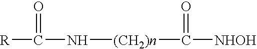

wherein R is a substituted or unsubstituted phenyl, piperidine, thiazole, 2-pyridine, 3-pyridine or 4-pyridine and n is an integer from about 4 to about 8; and

A compound represented by the structure:

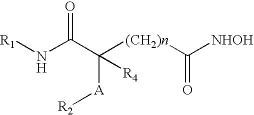

wherein A is an amide moiety, R₁ and R₂ are each selected from substituted or unsubstituted aryl, arylamino (e.g., pyridineamino, 9-purine-6-amine or thiazoleamino), arylalkyl, aryloxy, arylalkyloxy, R₄ is hydrogen, a halogen, a phenyl or a cycloalkyl group and n is an integer from about 3 to about 10.

The combination therapy can provide a therapeutic advantage in view of the differential toxicity associated with the two treatment modalities. More specifically, treatment with HDAC inhibitors can lead to hematologic toxicity, whereas radiotherapy can be toxic to tissue adjacent to the tumor site. As such, this differential toxicity can permit each treatment to be administered at its therapeutic dose, without increasing patient morbidity. Surprisingly however, the therapeutic effects achieved as a result of the combination treatment are enhanced or synergistic, for example, significantly better than additive therapeutic effects.

### BRIEF DESCRIPTION OF THE DRAWINGS

FIGS. 1A-D are plots of spheroid volume for LNCaP cells (A) untreated; (B) treated with 1 μM SAHA: (C) treated with 2.5 μM SAHA; and (D) treated with 5 μM SAHA for both continuous and 120 hour treatment times. The thick solid lines correspond to the median plot for each individual experiment.

FIGS. 2A-B are scans of light microscope images of the spheroids of LNCaP cells taken at different times after the start of continuous incubation with (A) 5 μM SAHA and (B) 2.5 μM SAHA (plots 1D and 1C above). Numbers on the bottom left of each panel correspond to time post-incubation in days.

FIGS. 3A-D are plots of median (thick lines) and individual (thin lines) spheroid volume for LNCaP cells treated according to the following regimen: A) untreated; B) incubated for 96 h with 5 μM SAHA; C) irradiated with an acute dose of external beam radiation using 6 Gy of Cs-137 irradiator (LET 02. keV/μm); and D) treated with 5 μM SAHA for 96 hours and an acute dose of radiation using 6 Gy of Cs-137 irradiator (LET 02. keV/μm) following at the midpoint (after 48 hours) of SAHA treatment.

FIG. 4 is a scan of light microscope images of a spheroid treated with the combination of SAHA and 6 Gy irradiation described in FIG. 3D. Numbers on the bottom left of each panel correspond to time from onset of incubation with SAHA.

FIGS. 5A-C are scans of TUNEL-stained sections of treated LNCaP spheroids. Panels (A-C) have been treated with SAHA alone (5 μM, 96 h). Panel (A) shows treated spheroids immediately following the end of incubation; Panel (B) shows treated spheroids 24 hours following the end of incubation with SAHA; Panel (C) shows treated spheroids 48 hours following the end of incubation with SAHA. Panels (D-F) show TUNEL staining for LNCaP spheroids treated with the combination SAHA+6 Gy radiation: Panel (D) is immediately after the end of incubation; Panel (E) is 24 hours following the end of incubation; and Panel (F) is 48 hours after the end of incubation. TUNEL staining for: Panel (G) a positive DNase treated control; Panel (H) an untreated spheroid; and Panel (I) a spheroid treated with 6 GY radiation, are also shown. All sections were counterstained with Haematoxylin.

FIGS. 6A-C are scans of Ki67-stained sections of treated LNCaP spheroids. Panels (A-C) have been treated with SAHA alone (5 μM, 96 h). Panel (A) shows spheroids immediately after the end of incubation with SAHA; Panel (B) shows spheroids 24 hour after the end of incubation; and Panel (C) shows spheroids 48 hours after the end of incubation. Panels D through F show Ki67 staining for spheroids treated with the combination SAHA+6 Gy radiation (D) immediately; (E) 24 hours and (F) 48 hours after the end of incubation. Ki67 staining for an untreated spheroid (G) and a spheroid treated with 6 Gy radiation (H) are also shown. All sections were counterstained with Haematoxylin.

FIGS. 7A-B are graphs showing the average and standard deviation of the percent positively stained cells for (A) TUNEL and (B) Ki67 staining. Three to five different sections were scored per experiment. The percentage of positively stained cells in SAHA-only sections versus SAHA+radiation was significantly different for Ki67 staining at 48 hours (p<0.01).

FIG. 8 is a graph showing spheroid volume for LNCaP cells treated according to the following regimen: ♦untreated control; ▪treated with Ac225-HuM 195; ▴treated for 96 h with 5 μM SAHA; X treated with Ac225-HuM 195 and 5 μM SAHA.

The foregoing and other objects, features and advantages of the invention will be apparent from the following more particular description of preferred embodiments of the invention, as illustrated in the accompanying drawings.

### DETAILED DESCRIPTION OF THE INVENTION

The present invention relates to a method for the treatment of cancer in a patient in need thereof. The method comprises administering to a patient in need thereof a first amount of a histone deacetylase inhibitor and a second amount or dose of radiation in a second treatment procedure. The first and second amounts together comprise a therapeutically effective amount.

In one embodiment, the method provides an anticancer effect which is synergistic.

Treatment of cancer, as used herein, refers to partially or totally inhibiting, delaying or preventing the progression of cancer including cancer metastasis; inhibiting, delaying or preventing the recurrence of cancer including cancer metastasis; or preventing the onset or development of cancer (chemoprevention) in a mammal, for example a human.

In one embodiment, the HDAC inhibitor sensitizes cancer cells in the patient to radiation. As such, the HDAC inhibitor can act as a radiosensitizer. For example, without wishing to be bound to any particular mechanism or theory, the therapeutic effect of the combination administration of an HDAC inhibitor and a radiation treatment can be due to the ability of the HDAC inhibitor to act as a radiosensitizer, thereby increasing the sensitivity of cancer cells in the patients to the radiation treatment. As such, the HDAC inhibitor can be administered in a radiosensitizing amount. The sensitization can be due to an irreversible arrest in cell cycling.

In another embodiment, the radiation sensitizes cancer cells in the patient to the action of the HDAC inhibitor.

The invention also relates to a method of determining the sensitivity of a particular cancer to the combination therapy of the invention. The method comprises exposing or contacting a cancer cell with a first amount of a histone deacetylase inhibitor in a first treatment procedure, and a second amount or dose of radiation in a second treatment procedure and assessing the anticancer effects. The first and second amounts together comprise a therapeutically effective amount. The anticancer effects can be assessed using any suitable assay.

In a further embodiment, the invention relates to a method of screening to determine optimum combinations of HDAC inhibitors and radiation therapy for particular cancer types. The method of screening comprises exposing a cancer cell to a first amount of a histone deacetylase inhibitor in a first treatment procedure, and a second amount or dose of radiation in a second treatment procedure. The first and second treatments together comprise a therapeutically effective amount. The cell can be in culture or present in the body of the patient in need of treatment. The anticancer effects of the treatment can be assessed using suitable methods.

As used herein the term “therapeutically effective amount” is intended to qualify the combined amount of the first and second treatments in the combination therapy. The combined amount will achieve the desired biological response. In the present invention, the desired biological response is partial or total inhibition, delay or prevention of the progression of cancer including cancer metastasis; inhibition, delay or prevention of the recurrence of cancer including cancer metastasis; or the prevention of the onset or development of cancer (chemoprevention) in a mammal, for example a human.

The combination therapy of the present invention is suitable for use in the treatment of a wide variety of cancers. As used herein, cancer refers to tumors, neoplasms, carcinomas, sarcomas, leukemias, lymphomas and the like. For example, cancers include, but are not limited to, leukemias and lymphomas such as cutaneous T-cell lymphoma (CTCL), noncutaneous peripheral T-cell lymphoma, lymphomas associated with human T-cell lymphotropic virus (HTLV), for example, adult T-cell leukemia/lymphoma (ATLL), acute lymphocytic leukemia, acute nonlymphocytic leukemias, chronic lymphocytic leukemia, chronic myelogenous leukemia, Hodgkin's Disease, non-Hodgkin's lymphomas, and multiple myeloma, childhood solid tumors such as brain tumors, neuroblastoma, retinoblastoma, Wilms' Tumor, bone tumors, and soft-tissue sarcomas, common solid tumors of adults such as head and neck cancers (e.g., oral, laryngeal and esophageal), genitourinary cancers (e.g., prostate, bladder, renal, uterine, ovarian, testicular, rectal and colon), lung cancer, breast cancer, pancreatic cancer, melanoma and other skin cancers, stomach cancer, brain cancer, liver cancer and thyroid cancer.

Histone Deacetylases and Histone Deacetylase Inhibitors

Histone deacetylases (HDACs) as that term is used herein are enzymes which catalyze the removal of acetyl groups from lysine residues in the amino terminal tails of the nucleosomal core histones. As such, HDACs together with histone acetyl transferases (HATs) regulate the acetylation status of histones. Histone acetylation affects gene expression and inhibitors of HDACs, such as the hydroxamic acid-based hybrid polar compound suberoylanilide hydroxamic acid (SAHA) induce growth arrest, differentiation and/or apoptosis of transformed cells in vitro and inhibit tumor growth in vivo. HDACs can be divided into three classes based on structural homology. Class I HDACs (HDACs 1, 2, 3 and 8) bear similarity to the yeast RPD3 protein, are located in the nucleus and are found in complexes associated with transcriptional co-repressors. Class II HDACs (HDACs 4, 5, 6, 7 and 9) are similar to the yeast HDA1 protein, and have both nuclear and cytoplasmic subcellular localization. Both Class I and II HDACs are inhibited by hydroxamic acid-based HDAC inhibitors, such as SAHA. Class III HDACs form a structurally distant class of NAD dependent enzymes that are related to the yeast SIR2 proteins and are not inhibited by hydroxamic acid-based HDAC inhibitors.

Histone deacetylase inhibitors or HDAC inhibitors, as that term is used herein are compounds which are capable of inhibiting the deacetylation of histones in vivo, in vitro or both. As such, HDAC inhibitors inhibit the activity of at least one histone deacetylase. As a result of inhibiting the deacetylation of at least one histone, an increase in acetylated histone occurs and accumulation of acetylated histone is a suitable biological marker for assessing the activity of HDAC inhibitors. Therefore, procedures which can assay for the accumulation of acetylated histones can be used to determine the HDAC inhibitory activity of compounds of interest. It is understood that compounds which can inhibit histone deacetylase activity can also bind to other substrates and as such can inhibit other biologically active molecules such as enzymes.

For example, in patients receiving HDAC inhibitors, the accumulation of acetylated histones in peripheral mononuclear cells as well as in tissue treated with HDAC inhibitors can be determined against a suitable control.

HDAC inhibitory activity of a particular compound can be determined in vitro using, for example, an enzymatic assays which shows inhibition of at least one histone deacetylase. Further, determination of the accumulation of acetylated histones in cells treated with a particular composition can be determinative of the HDAC inhibitory activity of a compound.

Assays for the accumulation of acetylated histones are well known in the literature. See, for example, Marks, P. A. et al., J. Natl. Cancer Inst., 92:1210-1215, 2000, Butler, L. M. et al., Cancer Res. 60:5165-5170 (2000), Richon, V. M. et al., Proc. Natl. Acad. Sci., USA, 95:3003-3007, 1998, and Yoshida, M. et al., J. Biol. Chem., 265:17174-17179, 1990.

For example, an enzymatic assay to determine the activity of a histone deacetylase inhibitor compound can be conducted as follows. Briefly, the effect of an HDAC inhibitor compound on affinity purified human epitope-tagged (Flag) HDAC1 can be assayed by incubating the enzyme preparation in the absence of substrate on under suitable temperatures for about 20 minutes with the indicated amount of inhibitor compound. Substrate ([³H]acetyl-labelled murine erythroleukemia cell-derived histone) can be added and the sample can be incubated for 20 minutes at about 37° C. in a total volume of 30 μL. The reaction can then be stopped and released acetate can be extracted and the amount of radioactivity released determined by scintillation counting. An alternative assay useful for determining the activity of a histone deacetylase inhibitor compound is the “HDAC Fluorescent Activity Assay; Drug Discovery Kit-AK-500” available from BIOMOL® Research Laboratories, Inc., Plymouth Meeting, Pa.

In vivo studies can be conducted as follows. Animals, for example mice, can be injected intraperitoneally with an HDAC inhibitor compound. Selected tissues, for example brain, spleen, liver etc, can be isolated at predetermined times, post administration. Histones can be isolated from tissues essentially as described by Yoshida et al., J. Biol. Chem. 265:17174-17179, 1990. Equal amounts of histones (about 1 μg) can be electrophoresed on 15% SDS-polyacrylamide gels and can be transferred to Hybond-P filters (available from Amersham). Filters can be blocked with 3% milk and can be probed with a rabbit purified polyclonal anti-acetylated histone H4 antibody (αAc-H4) and anti-acetylated histone H3 antibody (αAc-H3) (Upstate Biotechnology, Inc.). Levels of acetylated histone can be visualized using a horseradish peroxidase-conjugated goat anti-rabbit antibody (1:5000) and the SuperSignal chemiluminescent substrate (Pierce). As a loading control for the histone protein, parallel gels can be run and stained with Coomassie Blue (CB).

In addition, hydroxamic acid-based HDAC inhibitors like SAHA have been shown to up regulate the expression of the p21^{WAF1} gene, responsible for the inhibition of cyclin-dependent kinases that contributes to a transient arrest in the G₁ phase of the cell-cycle (Richon, V. M. et al. Proc Natl Acad Sci USA. 97: 10014-9., 2000). The p21^{WAF1} protein is induced within 2 hours of culture with HDAC inhibitors in a variety of transformed cells using standard methods. The induction of the p21^{WAF1} gene is associated with accumulation of acetylated histones in the chromatin region of this gene. Induction of p21^{WAF1} can therefore be recognized as involved in the G₁ cell cycle arrest caused by HDAC inhibitors in transformed cells.

Recently it has been shown that HDAC inhibitors like SAHA up-regulate thioredoxin-binding protein-2 (Butler, L. M. et al. Proc Natl Acad Sci USA. 99: 11700-5., 2002). TBP-2 is involved in the regulation of thioredoxin (Nishiyama, A. et al. J Biol Chem. 274: 21645-50., 1999). It inhibits the thiol reducing activity and reduces the level of thioredoxin. Thioredoxin is a major cellular protein disulfide reductase (Arner, E. S. et al. Eur J Biochem. 267: 6102-9., 2000). In addition to a number of other functions (Gasdaska, J. R. et al. Cell Growth Differ. 6: 1643-50., 1995; Berggren, M. et al. Anticancer Res. 16: 3459-66., 1996; Gallegos, A. et al. Cancer Res. 56: 5765-70., 1996; Grogan, T. M. et al. Hum Pathol. 31: 475-81., 2000; Baker, A. et al. Cancer Res. 57: 5162-7., 1997), thioredoxin serves as an electron donor in the ribonucleotide reductase reaction that is responsible for the reduction of nucleoside triphosphates to deoxynucleoside triphosphates needed in DNA replication and repair (Amer, E. S. et al. Eur J Biochem. 267: 6102-9., 2000). Like glutathione, thioredoxin is also a reducing agent involved in detoxification reactions and in the elimination of radiation-induced reactive oxygen species and other free radicals (Didier, C. et al. P Radic Biol Med. 30: 537-46., 2001).

As such, hydroxamic acid derivatives, such as SAHA, are suitable for use in treating or preventing a wide variety of thioredoxin (TRX)-mediated diseases and conditions, such as inflammatory diseases, allergic diseases, autoimmune diseases, diseases associated with oxidative stress or diseases characterized by cellular hyperproliferation (U.S. application No. Ser. 10/369,094, filed Feb. 15, 2003, entitled, “Method of treating TRX-mediated diseases using histone deacetylase inhibitors” by Richon et al., the entire content of which is hereby incorporated by reference).

Further, hydroxamic acid derivatives, such as SAHA, have recently been shown to be useful for treating diseases of the central nervous system (CNS), such as neurodegenerative diseases and for treating brain cancer (U.S. application Ser. No. 10/273,401, filed Oct. 16, 2002, entitled “Treatment of neurodegenerative diseases and cancer of the brain using histone deacetylase inhibitors” by Richon et al., the entire content of which is hereby incorporated by reference).

Typically, HDAC inhibitors fall into five general classes: 1) hydroxamic acid derivatives; 2) Short-Chain Fatty Acids (SCFAs); 3) cyclic tetrapeptides; 4) benzamides; and 5) electrophilic ketones.

Thus, all HDAC inhibitor compounds are suitable for use in the present invention. For example, suitable HDAC inhibitors include 1) hydroxamic acid derivatives; 2) Short-Chain Fatty Acids (SCFAs); 3) cyclic tetrapeptides; 4) benzamides; 5) electrophilic ketones; and/or any other class of compounds capable of inhibiting histone deacetylase.

Examples of such HDAC inhibitors include, but are not limited to:

A) HYDROXAMIC ACID DERIVATIVES such as Suberoylanilide Hydroxamic Acid (SAHA) (Richon et al., Proc. Natl. Acad. Sci. USA 95,3003-3007 (1998)); M-Carboxycinnamic Acid Bishydroxamide (CBHA) (Richon et al., supra); pyroxamide; CBHA; Trichostatin analogues such as Trichostatin A (TSA) and Trichostatin C (Koghe et al. 1998. Biochem. Pharmacol. 56: 1359-1364); Salicylihydroxamic Acid (SBHA) (Andrews et al., International J. Parasitology 30,761-768 (2000)); Azelaic Bishydroxamic Acid (ABHA) (Andrews et al., supra); Azelaic-1-Hydroxamate-9-Anilide (AAHA) (Qiu et al., Mol. Biol. Cell 11, 2069-2083 (2000)); 6-(3-Chlorophenylureido) carpoic Hydroxamic Acid (3Cl-UCHA), Oxamflatin [(2E)-5-[3-[(phenylsuibnyl)amino phenyl]-pent-2-en-4-ynohydroxamic acid (Kim et al. Oncogene, 18: 2461 2470 (1999)); A-161906, Scriptaid (Su et al. 2000 Cancer Research, 60: 3137-3142); PXD-101 (Prolifix); LAQ-824; CHAP; MW2796 (Andrews et al., supra); and MW2996 (Andrews et al., supra).

B) CYCLIC TETRAPEPTIDES such as Trapoxin A (TPX)-Cyclic Tetrapeptide (cyclo-(L-phenylalanyl-L-phenylalanyl-D-pipecolinyl-L-2-amino-8-oxo-9,10-epoxy decanoyl)) (Kijima et al., J Biol. Chem. 268,22429-22435 (1993)); FR901228 (FK 228, Depsipeptide) (Nakajima et al., Ex. Cell Res. 241,126-133 (1998)); FR225497 Cyclic Tetrapeptide (H. Mori et al., PCT Application WO 00/08048 (Feb. 17, 2000));, Apicidin Cyclic Tetrapeptide [cyclo (NO-methyl-L-tryptophanyl-L-isoleucinyl-D-pipecolinyl-L-2-amino-8oxodecanoyl)] (Darkin-Rattray et al., Proc. Natl. Acad. Sci. USA 93,1314313147 (1996)); Apicidin la, Apicidin Ib, Apicidin Ic, Apicidin IIa, and Apicidin IIb (P. Dulski et al., PCT Application WO 97/11366); CHAP, HC-Toxin Cyclic Tetrapeptide (Bosch et al., Plant Cell 7, 1941-1950 (1995)); WF27082 Cyclic Tetrapeptide (PCT Application WO 98/48825); and Chiamydocin (Bosch et al., supra).

C) SHORT CHAIN FATTY ACID (SCFA) DERIVATIVES such as: Sodium Butyrate (Cousens et al., J. Biol. Chem. 254,1716-1723 (1979)); Isovalerate (McBain et al., Biochem. Pharm. 53: 1357-1368 (1997)); Valerate (McBain et al., supra); 4 Phenylbutyrate (4-PBA) (Lea and Tulsyan, Anticancer Research, 15,879-873 (1995)); Phenylbutyrate (PB) (Wang et al., Cancer Research, 59, 2766-2799 (1999)); Propionate (McBain et al., supra); Butyramide (Lea and Tulsyan, supra); Isobutyramide (Lea and Tulsyan, supra); Phenylacetate (Lea and Tulsyan, supra); 3-Bromopropionate (Lea and Tulsyan, supra); Tributyrin (Guan et al., Cancer Research, 60,749-755 (2000)); Valproic acid and Valproate.

D) BENZAMIDE DERIVATIVES such as CI-994; MS-27-275 [N-(2-aminophenyl)-4-[N-(pyridin-3-ylmethoxycarbonyl)aminomethyl]benzamide] (Saito et al., Proc. Natl. Acad. Sci. USA 96, 4592-4597 (1999)); and 3′-amino derivative of MS-27-275 (Saito et al., supra).

E) ELECTROPHILIC KETONE DERIVATIVES such as trifluoromethyl ketones (Frey et al, Bioorganic & Med. Chem. Lett. (2002), 12, 3443-3447; U.S. 6,511,990) and α-keto amides such as N-methyl-α-ketoamides

F) OTHER HDAC Inhibitors such as Depudecin (Kwon et al. 1998. PNAS 95: 3356-3361.

Preferred hydroxamic acid based HDAC inhibitors are suberoylanilide hydroxamic acid (SAHA), m-carboxycinnamic acid bishydroxamate (CBHA) and pyroxamide. SAHA has been shown to bind directly in the catalytic pocket of the histone deacetylase enzyme. SAHA induces cell cycle arrest, differentiation and/or apoptosis of transformed cells in culture and inhibits tumor growth in rodents. SAHA is effective at inducing these effects in both solid tumors and hematological cancers. It has been shown that SAHA is effective at inhibiting tumor growth in animals with no toxicity to the animal. The SAHA-induced inhibition of tumor growth is associated with an accumulation of acetylated histones in the tumor. SAHA is effective at inhibiting the development and continued growth of carcinogen-induced (N-methylnitrosourea) mammary tumors in rats. SAHA was administered to the rats in their diet over the 130 days of the study. Thus, SAHA is a nontoxic, orally active antitumor agent whose mechanism of action involves the inhibition of histone deacetylase activity.

SAHA can be represented by the following structural formula:

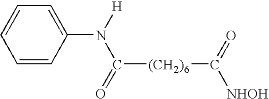

Pyroxamide can be represented by the following structural formula:

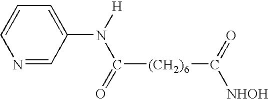

CBHA can be represented by the structural formula:

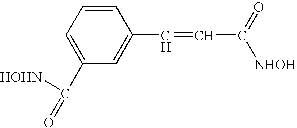

In one embodiment, the HDAC inhibitor can be represented by Formula I:

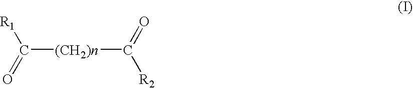

wherein R₁ and R₂ can be the same or different; when R₁ and R₂ are the same, each is a substituted or unsubstituted arylamino (e.g., pyridineamino, 9-purine-6-amino or thiazoleamino), cycloalkylamino or piperidino group; when R₁ and R₂ are different R₁═R₃—N—R₄, wherein each of R₃ and R₄ are independently the same as or different from each other and are a hydrogen atom, a hydroxyl group, a substituted or unsubstituted, branched or unbranched alkyl, alkenyl, cycloalkyl, aryl, alkyloxy, aryloxy, arylalkyloxy group, or R₃ and R₄ are bonded together to form a piperidine group, R₂ is a hydroxylamino, hydroxyl, amino, alkylamino, dialkylamino or alkyloxy group and n is an integer from about 4 to about 8.

As such, in another embodiment the HDAC inhibitors used in the method of the invention can be represented by Formula II:

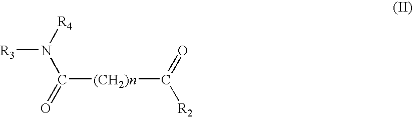

wherein each of R₃ and R₄ are independently the same as or different from each other and are a hydrogen atom, a hydroxyl group, a substituted or unsubstituted, branched or unbranched alkyl, alkenyl, cycloalkyl, aryl, alkyloxy, aryloxy or arylalkyloxy group, or R₃ and R₄ are bonded together to form a piperidine group, R₂ is a hydroxylamino, hydroxyl, amino, alkylamino, dialkylamino or alkyloxy group and n is an integer from about 4 to about 8.

In a particular embodiment of Formula II, R₂ is a hydroxylamino, hydroxyl, amino, methylamino, dimethylamino or methyloxy group and n is 6. In yet another embodiment of Formula II, R₄is a hydrogen atom, R₃ is a substituted or unsubstituted phenyl and n is 6. In further embodiments of Formula II, R₄ is hydrogen and R₃ is an α-, β-, or γ-pyridine.

In other specific embodiments of Formula II, R₄ is a hydrogen atom and R₃ is a cyclohexyl group; R₄ is a hydrogen atom and R₃ is a methoxy group; R₃ and R₄ each bond together to form a piperidine group; R₄ is a hydrogen atom and R₃ is a hydroxyl group; R₃ and R₄ are both a methyl group and R₃ is phenyl and R₄ is methyl.

Further HDAC inhibitors suitable for use in the present invention can be represented by structural Formula III:

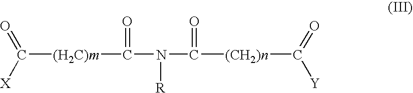

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino, or aryloxyalkylamino group; R is a hydrogen atom, a hydroxyl, group, a substituted or unsubstituted alkyl, arylalkyloxy, or aryloxy group; and each of m and n are independently the same as or different from each other and are each an integer from about 0 to about 8.

In a particular embodiment, the HDAC inhibitor is a compound of Formula III wherein X, Y and R are each hydroxyl and both m and n are 5.

In yet another embodiment, the HDAC inhibitor compounds suitable for use in the method of the invention can be represented by structural Formula IV:

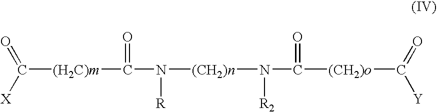

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; each of R₁ and R₂ are independently the same as or different from each other and are a hydrogen atom, a hydroxyl group, a substituted or unsubstituted alkyl, aryl, alkyloxy, or aryloxy group; and each of m, n and o are independently the same as or different from each other and are each an integer from about 0 to about 8.

Other HDAC inhibitors suitable for use in the invention include compounds having structural Formula V:

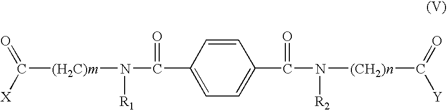

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; each of R₁ and R₂ are independently the same as or different from each other and are a hydrogen atom, a hydroxyl group, a substituted or unsubstituted alkyl, aryl, alkyloxy, or aryloxy group; and each of m and n are independently the same as or different from each other and are each an integer from about 0 to about 8.

In a further embodiment, HDAC inhibitors suitable for use in the method of the present invention can have structural Formula VI:

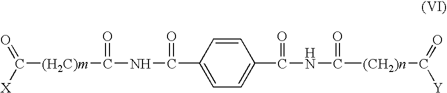

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; and each of m and n are independently the same as or different from each other and are each an integer from about 0 to about 8.

In yet another embodiment, the HDAC inhibitors useful in the method of the invention can have structural Formula VII:

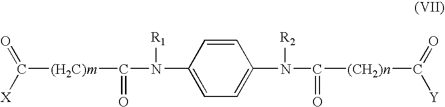

wherein each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; R₁ and R₂ are independently the same as or different from each other and are a hydrogen atom, a hydroxyl group, a substituted or unsubstituted alkyl, arylalkyloxy or aryloxy group; and each of m and n are independently the same as or different from each other and are each an integer from about 0 to about 8.

In yet a further embodiment, HDAC inhibitors suitable for use in the invention can have structural Formula VIII:

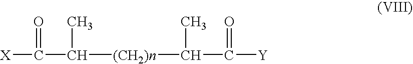

wherein each of X an Y are independently the same as or different from each other and are a hydroxyl, amino or hydroxylamino group, a substituted or unsubstituted alkyloxy, alkylamino, dialkylamino, arylamino, alkylarylamino, or aryloxyalkylamino group; and n is an integer from about 0 to about 8.

Additional compounds suitable for use in the method of the invention include those represented by Formula IX:

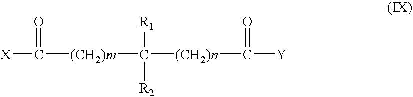

wherein Each of X and Y are independently the same as or different from each other and are a hydroxyl, amino or hydroxylamino group, a substituted or unsubstituted alkyloxy, alkylamino, dialkylamino, arylamino, alkylarylamino, alkyloxyamino, aryloxyamino, alkyloxyalkylamino or aryloxyalkylamino group; each of R₁ and R₂ are independently the same as or different from each other and are a hydrogen atom, a hydroxyl group, a substituted or unsubstituted alkyl, aryl, alkyloxy, aryloxy, carbonylhydroxylamino or fluoro group; and each of m and n are independently the same as or different from each other and are each an integer from about 0 to about 8.

In a further embodiment, HDAC inhibitors suitable for use in the invention include compounds having structural Formula X:

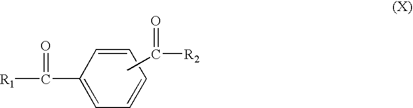

wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group. In a particular embodiment, the HDAC inhibitor is a compound of structural Formula X wherein R₁ and R₂ are both hydroxylamino. In a further embodiment, the HDAC inhibitor suitable for use in the invention has structural Formula XI:

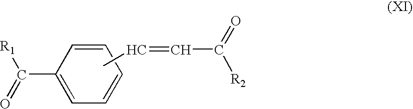

wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group. In a particular embodiment, the HDAC inhibitor is a compound of structural Formula XI wherein R₁ and R₂ are both hydroxylamino.

In a further embodiment, HDAC inhibitors suitable for use in the present invention include compounds represented by structural Formula XII:

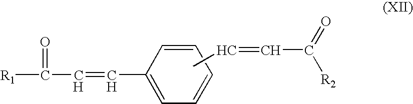

wherein each of R₁ and R₂ are independently the same as or different from each other and are a hydroxyl, alkyloxy, amino, hydroxylamino, alkylamino, dialkylamino, arylamino, alkylarylamino, alkyloxyamino, aryloxyamino, alkyloxyalkylamino, or aryloxyalkylamino group. In a particular embodiment, the HDAC inhibitor is a compound of structural Formula XII wherein R₁ and R₂ are both hydroxylamino.

Additional compounds suitable for use in the method of the invention include those represented by structural Formula XIII:

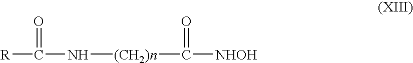

wherein R is a substituted or unsubstituted phenyl, piperidine, thiazole, 2-pyridine, 3-pyridine or 4-pyridine and n is an integer from about 4 to about 8.

In yet another embodiment, the HDAC inhibitors suitable for use in the method of the invention can be represented by structural Formula (XIV):

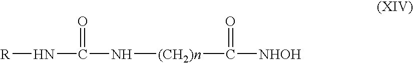

wherein R is a substituted or unsubstituted phenyl, pyridine, piperidine or thiazole group and n is an integer from about 4 to about 8 or a pharmaceutically acceptable salt thereof.

In a particular embodiment, R is phenyl and n is 5. In another embodiment, n is 5 and R is 3-chlorophenyl.

Other HDAC inhibitors useful in the present invention can be represented by structural Formula XV:

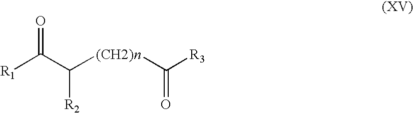

wherein each of R₁ and R₂ is directly attached or through a linker and is a hydroxyl, substituted or unsubstituted, aryl (e.g. naphthyl, phenyl, quinolinyl, isoquinolinyl or pyridyl), cycloalkyl, cycloalkylamino, piperidino, branched or unbranched alkyl, alkenyl, arylamino (pyridineamino, 9-purine-6-amino or thiazoleamino), arylalkylamino, arylalkyl, alkyloxy, aryloxy or arylalkoxy group; n is an integer from about 3 to about 10 and R₃ is a hydroxamic acid, hydroxylamino, hydroxyl, amino, alkylamino or alkyloxy group.

The linker can be an amide moiety, —O—, —S—, —NH— or —CH2—.

In certain embodiments, R₁ is —NH—R₄ wherein R₄ is a hydroxyl, substituted or unsubstituted, aryl (e.g., naphthyl, phenyl, quinolinyl, isoquinolinyl or pyridyl), cycloalkyl, cycloalkylamino, piperidino, branched or unbranched alkyl, alkenyl, arylamino (e.g., pyridineamino, 9-purine-6-amine or thiazoleamino), arylalkylamino, alkyloxy, arylalkyl, aryloxy or arylalkyloxy group.

Further and more specific HDAC inhibitors of Formula XV, include those which can be represented by Formula XVI:

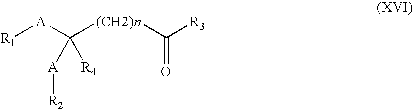

wherein each of R₁ and R₂ is hydroxyl, substituted or unsubstituted, aryl (e.g., phenyl, naphthyl, quinolinyl, isoquinolinyl or pyridyl), cycloalkyl, cycloalkylamino, piperidino, arylamino (e.g., pyridineamino, 9-purine-6-amino or thiazoleamino), arylalkylamino, branched or unbranched alkyl, alkenyl, alkyloxy, arylalkyl, aryloxy or arylalkyloxy group; R₃ is hydroxamic acid, hydroxylamino, hydroxyl, amino, alkylamino or alkyloxy group; R₄ is hydrogen, halogen, phenyl or a cycloalkyl moiety; and A can be the same or different and represents an amide moiety, —O—, —S—, —NR₅— or —CH₂— where R₅ is a substituted or unsubstituted C₁-C₅ alkyl and n is an integer from about 3 to about 10.

For example, further compounds having a more specific structure within Formula XVI can be represented by structural Formula XVII:

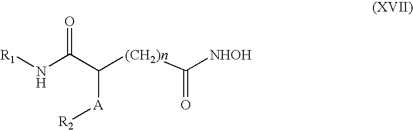

wherein A is an amide moiety, R₁ and R₂ are each selected from substituted or unsubstituted aryl (e.g., phenyl, naphthyl, quinolinyl, isoquinolinyl or pyridyl), arylamino (e.g., pyridineamino, 9-purine-6-amine or thiazoleamino), arylalkylamino, arylalkyl, aryloxy or arylalkyloxy group and n is an integer from about 3 to about 10.

For example, compounds having an amide moiety at A can be represented by the formula:

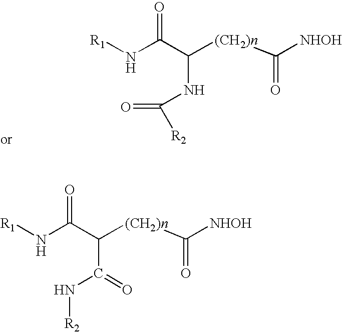

In another embodiment, the HDAC inhibitor can have the Formula XVIII:

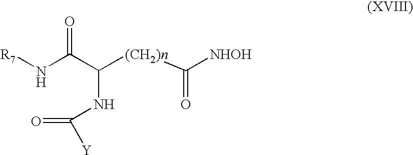

wherein R₇ is selected from substituted or unsubstituted aryl (e.g., phenyl, naphthyl, quinolinyl, isoquinolinyl or pyridyl), arylamino (e.g., pyridineamino, 9-purine-6-amine or thiazoleamino), arylalkylamino, arylalkyl, aryloxy or arylalkyloxy and n is an integer from about 3 to about 10 and Y is selected from

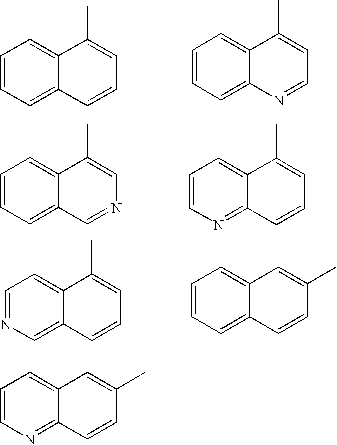

or a pharmaceutically acceptable salt thereof.

In a further embodiment, the HDAC inhibitor compound can have Formula XIX:

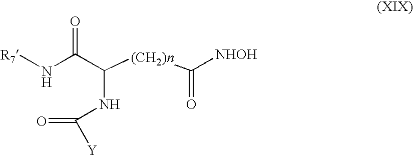

wherein n is an integer from about 3 to about 10, Y is selected from

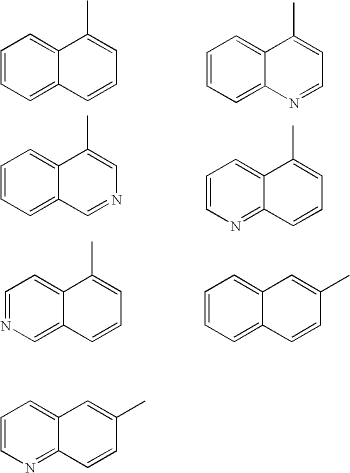

and R₇′ is selected from

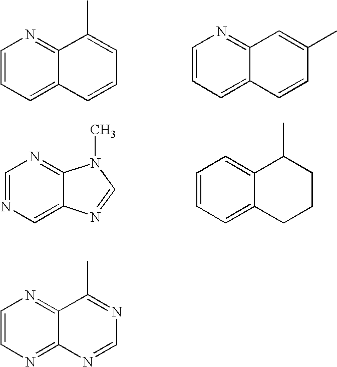

or a pharmaceutically acceptable salt thereof.

Further compounds for use in the invention can be represented by structural Formula XX:

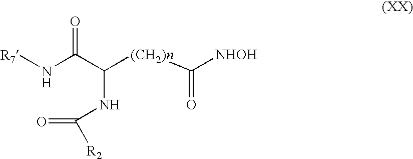

wherein R₂ is selected from a substituted or unsubstituted aryl, arylamino (e.g., pyridineamino, 9-purine-6-amino or thiazoleamino), arylalkylamino, arylalkyl or aryloxy, arylalkyloxy group and n is an integer from 3 to 10 and R₇′ is selected from

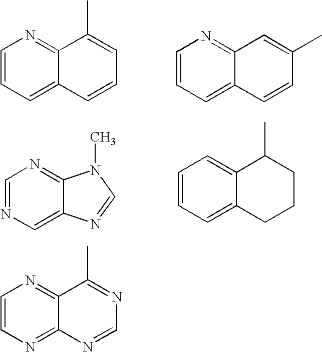

Further HDAC inhibitors useful in the invention can be represented by structural Formula XXI:

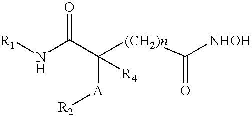

wherein A is an amide moiety, R₁ and R₂ are each selected from a substituted or unsubstituted aryl, arylamino (e.g., pyridineamino, 9-purine-6-amine or thiazoleamino) arylalkylamino, arylalkyl, aryloxy or arylalkyloxy group, R₄ is hydrogen, a halogen, a phenyl or a cycloalkyl moiety and n is an integer from about 3 to about 10 or a pharmaceutically acceptable salt thereof.

For example, a compound of Formula XXI can be represented by the structure:

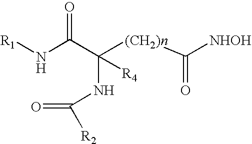

or can be represented by the structure:

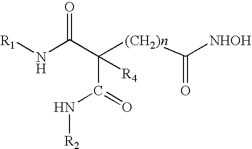

wherein R₁, R₂, R₄ and n have the meanings of Formula XXI. Further, HDAC inhibitors having the structural Formula XXII:

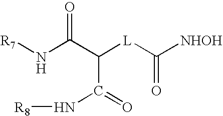

wherein L is a linker selected from the group consisting of —(CH2)ₙ—, —(CH═CH)ₘ, phenyl, -cycloalkyl-, or any combination thereof; and wherein each of R₇ and R₈ are independently substituted or unsubstituted, aryl, arylamino (e.g., pyridineamino, 9-purine-6-amino or thiazoleamino), arylalkylamino, arylalkyl, aryloxy or arylalkyloxy group, n is an integer from about 3 to about 10 and m is an integer from 0-10.

For example, a compound of Formula XXII can be:

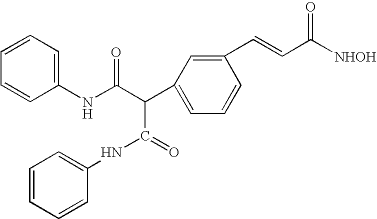

Other HDAC inhibitors suitable for use in the invention include those shown in the following more specific formulas:

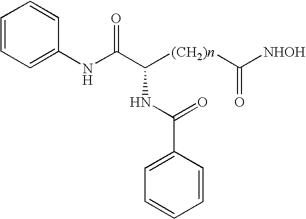

wherein n is an integer from 3 to 10 or an enantiomer or,

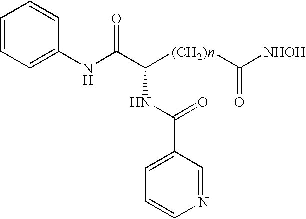

wherein n is an integer from 3 to 10 or an enantiomer or

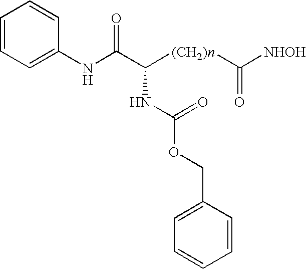

wherein n is an integer from 3 to 10 or an enantiomer or

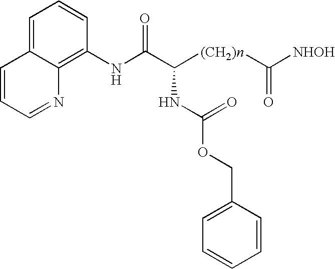

wherein n is an integer from 3 to 10 or an enantiomer or

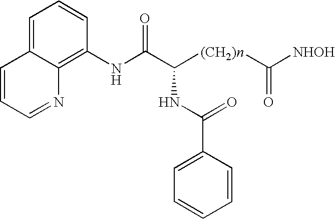

wherein n is an integer from 3 to 10 or an enantiomer.

Further specific HDAC inhibitors suitable for use in the invention include

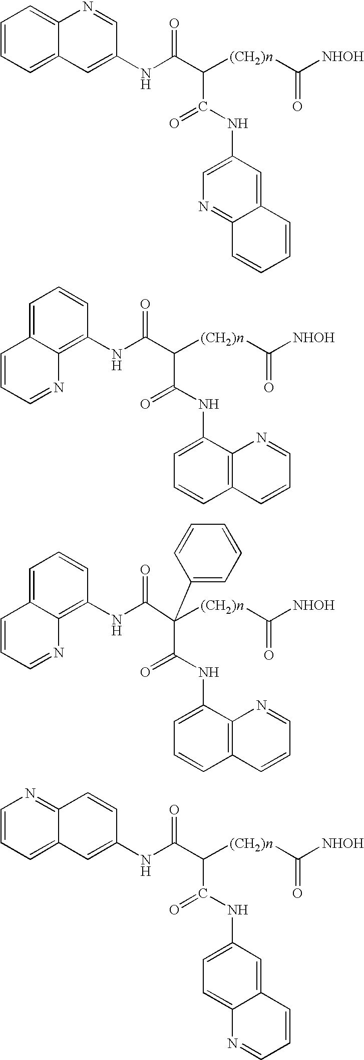

wherein n in each is an integer from 3 to 10 and the compound

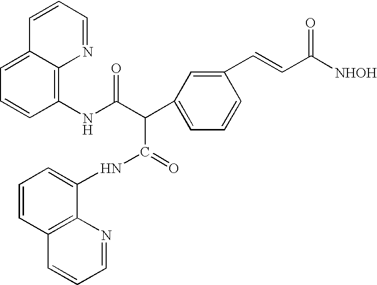

Further specific HDAC inhibitors of include those which can be represented by Formula XXIII:

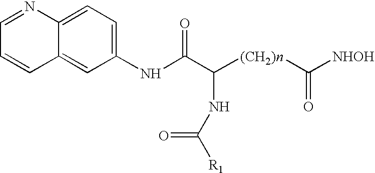

wherein R₁ is a substituted or unsubstituted aryl group, arylalkyl group, arylamino group, arylalkylamino group, aryloxy group or arylalkoxy group and n is an integer from 3 to 10. In a particular embodiment, n is 5 for the compounds of Structural Formula XXIII.

In a specific embodiment, the compound of Formula XXIII is represented by the following structure:

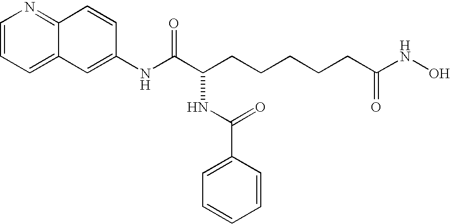

In another specific embodiment, the compound of Formula XXIII is represented by the following structure:

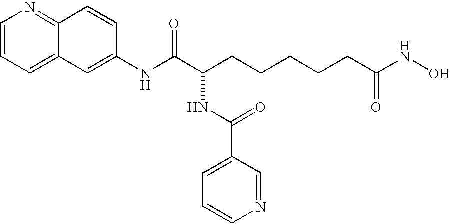

In yet another specific embodiment, the compound of Formula XXIII is represented by the following structure:

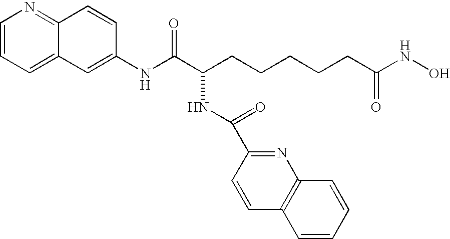

In still another specific embodiment, the compound of Formula XXIII is represented by the following structure:

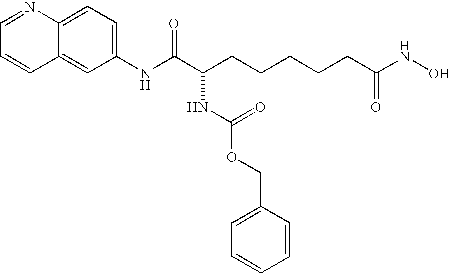

Further specific HDAC inhibitors include those which can be represented by Formula XXIV:

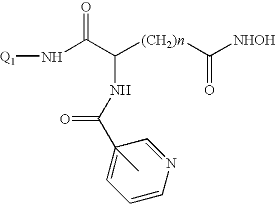

wherein Q₁ is a substituted or unsubstituted quinolinyl or isoquinolinyl group and n is an integer from 3 to 10. In a particular embodiment, n is 5 for the compounds of Structural Formula XXIV.

In a specific embodiment, the compound of Formula XXIV is represented by the following structure:

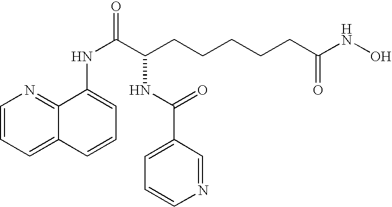

Further specific HDAC inhibitors include those which can be represented by Formula XXV:

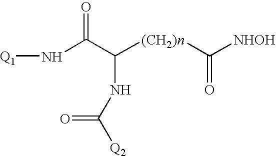

wherein Q₁ and Q₂ are independently a substituted or unsubstituted quinolinyl or isoquinolinyl group and n is an integer from about 3 to about 10. In a particular embodiment, n is 5 for the compounds of Structural Formula XXV.

In a specific embodiment, the compound of Formula XXV is represented by the following structure:

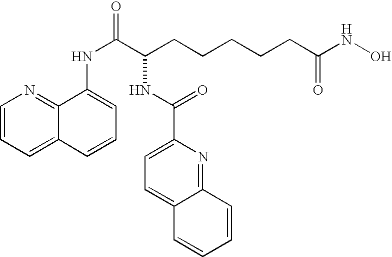

Further specific HDAC inhibitors include those which can be represented by Formula XXVI:

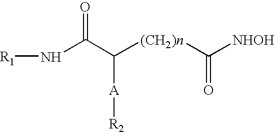

wherein R₁ is an arylalkyl, R₂ is a substituted or unsubstituted aryl group, arylalkyl group, arylamino group, arylalkylamino group, aryloxy group or arylalkoxy group, A is an amide and n is an integer from 3 to 10. In a particular embodiment, n is 5 for the compounds of Structural Formula XXVI.

In a specific embodiment, the compound of Formula XXVI is represented by the following structure:

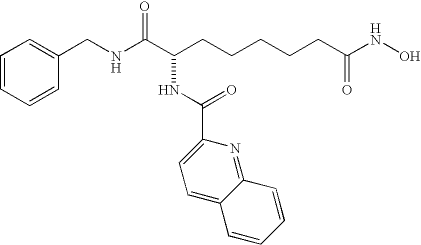

In a specific embodiment, the compound of Formula XXVI is represented by the following structure:

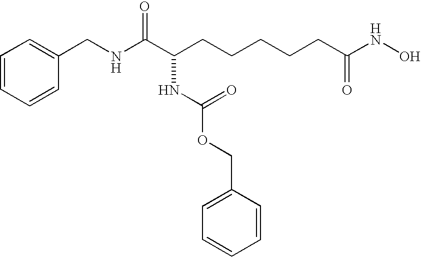

In a specific embodiment, the compound of Formula XXVI is represented by the following structure:

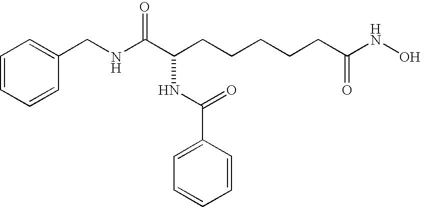

Other examples of such compounds and other HDAC inhibitors can be found in U.S. Pat. Nos. 5,369,108, issued on Nov. 29, 1994, 5,700,811, issued on Dec. 23, 1997, 5,773,474, issued on Jun. 30, 1998, 5,932,616 issued on Aug. 3, 1999 and 6,511,990, issued Jan. 28, 2003 all to Breslow et al.; U.S. Pat. Nos. 5,055,608, issued on Oct. 8, 1991, 5,175,191, issued on Dec. 29, 1992 and 5,608,108, issued on Mar. 4, 1997 all to Marks et al.; U.S. Provisional Application No. 60/459,826, filed Apr. 1, 2003 in the name of Breslow et al.; as well as, Yoshida, M., et al., Bioassays 17, 423-430 (1995); Saito, A., et al., PNAS USA 96, 4592-4597, (1999); Furamai R. et al., PNAS USA 98 (1), 87-92 (2001); Komatsu, Y., et al., Cancer Res. 61(11), 4459-4466 (2001); Su, G. H., et al., Cancer Res. 60, 3137-3142 (2000); Lee, B. I. et al., Cancer Res. 61(3), 931-934; Suzuki, T., et al., J. Med. Chem. 42(15), 3001-3003 (1999); published PCT Application WO 01/18171 published on Mar. 15, 2001 Sloan-Kettering Institute for Cancer Research and The Trustees of Columbia University; published PCT Application WO02/246144 to Hoffmann-La Roche; published PCT Application WO02/22577 to Novartis; published PCT Application WO02/30879 to Prolifix; published PCT Applications WO 01/38322 (published May 31, 2001), WO 01/70675 (published on Sep. 27, 2001) and WO 00/71703 (published on Nov. 30, 2000) all to Methylgene, Inc.; published PCT Application WO 00/21979 published on Oct. 8, 1999 to Fujisawa Pharmaceutical Co., Ltd.; published PCT Application WO 98/40080 published on Mar. 11, 1998 to Beacon Laboratories, L.L.C.; and Curtin M. (Current patent status of histone deacetylase inhibitors Expert Opin. Ther. Patents (2002) 12(9): 1375-1384 and references cited therein).

Specific non-limiting examples of HDAC inhibitors are provided in the Table below. It should be noted that the present invention encompasses any compounds which are structurally similar to the compounds represented below, and which are capable of inhibiting histone deacetylases.

Title

| | |
|---|---|
| MS-275 | 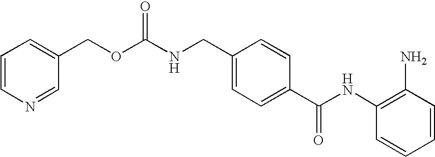 |
| DEPSIPEPTIDE | 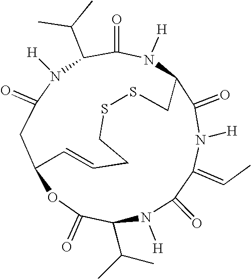 |
| CI-994 | 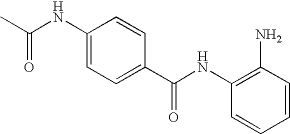 |
| Apicidin | 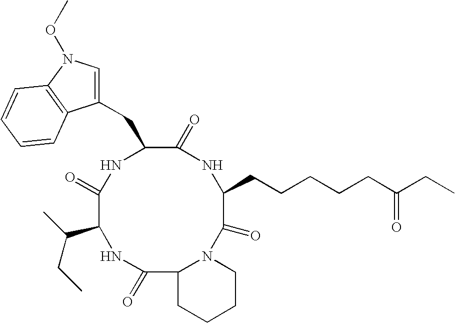 |
| A-161906 | 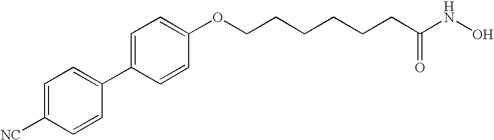 |
| Scriptaid | 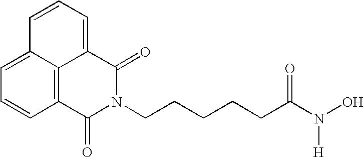 |
| PXD-101 | 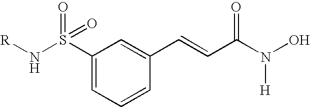 |
| CHAP | 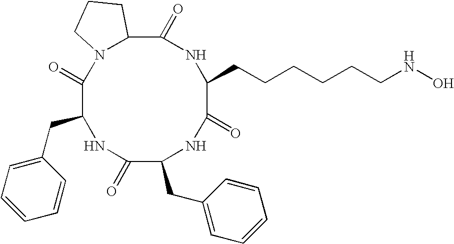 |
| LAQ-824 | 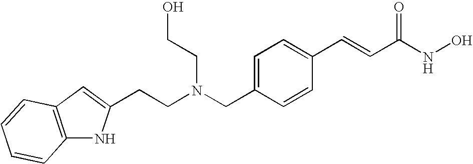 |
| Butyric Acid | 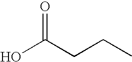 |
| Depudecin | 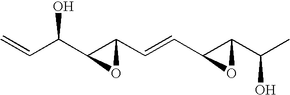 |
| Oxamflatin | 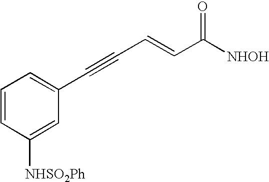 |
| Trichostatin C | 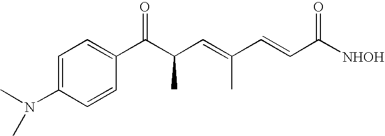 |

Definitions

An “aliphatic group” is non-aromatic, consists solely of carbon and hydrogen and can optionally contain one or more units of unsaturation, e.g., double and/or triple bonds. An aliphatic group can be straight chained, branched or cyclic. When straight chained or branched, an aliphatic group typically contains between about 1 and about 12 carbon atoms, more typically between about 1 and about 6 carbon atoms. When cyclic, an aliphatic group typically contains between about 3 and about 10 carbon atoms, more typically between about 3 and about 7 carbon atoms. Aliphatic groups are preferably C₁-C₁₂ straight chained or branched alkyl groups (i.e., completely saturated aliphatic groups), more preferably C₁-C₆ straight chained or branched alkyl groups. Examples include methyl, ethyl, n-propyl, iso-propyl, n-butyl, sec-butyl and tert-butyl.

An “aromatic group” (also referred to as an “aryl group”) as used herein includes carbocyclic aromatic groups, heterocyclic aromatic groups (also referred to as “heteroaryl”) and fused polycyclic aromatic ring system as defined herein.

A “carbocyclic aromatic group” is an aromatic ring of 5 to 14 carbons atoms, and includes a carbocyclic aromatic group fused with a 5-or 6-membered cycloalkyl group such as indan. Examples of carbocyclic aromatic groups include, but are not limited to, phenyl, naphthyl, e.g., 1-naphthyl and 2-naphthyl; anthracenyl, e.g., 1-anthracenyl, 2-anthracenyl; phenanthrenyl; fluorenonyl, e.g., 9-fluorenonyl, indanyl and the like. A carbocyclic aromatic group is optionally substituted with a designated number of substituents, described below.

A “heterocyclic aromatic group” (or “heteroaryl”) is a monocyclic, bicyclic or tricyclic aromatic ring of 5- to 14-ring atoms of carbon and from one to four heteroatoms selected from O, N, or S. Examples of heteroaryl include, but are not limited to pyridyl, e.g., 2-pyridyl (also referred to as “α-pyridyl), 3-pyridyl (also referred to as β-pyridyl) and 4-pyridyl (also referred to as (γ-pyridyl); thienyl, e.g., 2-thienyl and 3-thienyl; furanyl, e.g., 2-furanyl and 3-furanyl; pyrimidyl, e.g., 2-pyrimidyl and 4-pyrimidyl; imidazolyl, e.g., 2-imidazolyl; pyranyl, e.g., 2-pyranyl and 3-pyranyl; pyrazolyl, e.g., 4-pyrazolyl and 5-pyrazolyl; thiazolyl, e.g., 2-thiazolyl, 4-thiazolyl and 5-thiazolyl; thiadiazolyl; isothiazolyl; oxazolyl, e.g., 2-oxazoyl, 4-oxazoyl and 5-oxazoyl; isoxazoyl; pyrrolyl; pyridazinyl; pyrazinyl and the like. Heterocyclic aromatic (or heteroaryl) as defined above can be optionally substituted with a designated number of substituents, as described below for aromatic groups.

A “fused polycyclic aromatic” ring system is a carbocyclic aromatic group or heteroaryl fused with one or more other heteroaryl or nonaromatic heterocyclic ring. Examples include, quinolinyl and isoquinolinyl, e.g., 2-quinolinyl, 3-quinolinyl, 4-quinolinyl, 5-quinolinyl, 6-quinolinyl, 7-quinolinyl and 8-quinolinyl, 1-isoquinolinyl, 3-quinolinyl, 4-isoquinolinyl, 5-isoquinolinyl, 6-isoquinolinyl, 7-isoquinolinyl and 8-isoquinolinyl; benzofuranyl e.g., 2-benzofuranyl and 3-benzofuranyl; dibenzofuranyl. e.g., 2,3-dihydrobenzofuranyl; dibenzothiophenyl; benzothienyl, e.g., 2-benzothienyl and 3-benzothienyl; indolyl, e.g., 2-indolyl and 3-indolyl; benzothiazolyl, e.g., 2-benzothiazolyl; benzooxazolyl, e.g., 2-benzooxazolyl; benzimidazolyl, e.g., 2-benzoimidazolyl; isoindolyl, e.g., 1-isoindolyl and 3-isoindolyl; benzotriazolyl; purinyl; thianaphthenyl and the like. Fused polycyclic aromatic ring systems can optionally be substituted with a designated number of substituents, as described herein.

An “aralkyl group” (arylalkyl) is an alkyl group substituted with an aromatic group, preferably a phenyl group. A preferred aralkyl group is a benzyl group. Suitable aromatic groups are described herein and suitable alkyl groups are described herein. Suitable substituents for an aralkyl group are described herein.

An “aryloxy group” is an aryl group that is attached to a compound via an oxygen (e.g., phenoxy).

An “alkoxy group” (alkyloxy), as used herein, is a straight chain or branched C₁-C₁₂ or cyclic C₃-C₁₂ alkyl group that is connected to a compound via an oxygen atom. Examples of alkoxy groups include but are not limited to methoxy, ethoxy and propoxy.

An “arylalkoxy group” (arylalkyloxy) is an arylalkyl group that is attached to a compound via an oxygen on the alkyl portion of the arylalkyl (e.g., phenylmethoxy).

An “arylamino group” as used herein, is an aryl group that is attached to a compound via a nitrogen.

As used herein, an “arylalkylamino group” is an arylalkyl group that is attached to a compound via a nitrogen on the alkyl portion of the arylalkyl.

As used herein, many moieties or groups are referred to as being either “substituted or unsubstituted”. When a moiety is referred to as substituted, it denotes that any portion of the moiety that is known to one skilled in the art as being available for substitution can be substituted. For example, the substitutable group can be a hydrogen atom which is replaced with a group other than hydrogen (i.e., a substituent group). Multiple substituent groups can be present. When multiple substituents are present, the substituents can be the same or different and substitution can be at any of the substitutable sites. Such means for substitution are well-known in the art. For purposes of exemplification, which should not be construed as limiting the scope of this invention, some examples of groups that are substituents are: alkyl groups (which can also be substituted, with one or more substituents, such as CF₃), alkoxy groups (which can be substituted, such as OCF₃), a halogen or halo group (F, Cl, Br, I), hydroxy, nitro, oxo, —CN, —COH, —COOH, amino, azido, N-alkylamino or N,N-dialkylamino (in which the alkyl groups can also be substituted), esters (—C(O)—OR, where R can be a group such as alkyl, aryl, etc., which can be substituted), aryl (most preferred is phenyl, which can be substituted), arylalkyl (which can be substituted) and aryloxy.

Stereochemistry

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the formulas of the invention, it is understood that both the (R) and (S) configurations of the chiral carbon, and hence both enantiomers and mixtures thereof, are embraced within the formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines is (bonds to atoms below the plane). The Cahn-Inglod-Prelog system can be used to assign the (R) or (S) configuration to a chiral carbon.

When the HDAC inhibitors of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, for example by formation of diastereoisomer salts which can be separated, for example, by crystallization (See, CRC Handbook of Optical Resolutions via Diastereomeric Salt Formation by David Kozma (CRC Press, 2001)); formation of diastereoisomer derivatives or complexes which can be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ee) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. For example, the enantiomeric excess can be about 60% or more, such as about 70% or more, for example about 80% or more, such as about 90% or more. In a particular embodiment when a specific absolute configuration is designated, the enantiomeric excess of depicted compounds is at least about 90%. In a more particular embodiment, the enantiomeric excess of the compounds is at least about 95%, such as at least about 97.5%, for example, at least 99% enantiomeric excess.

When a compound of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers which are not mirror-images (e.g., (S,S) and (R,S)) are diastereomers. The diastereoisomer pairs can be separated by methods known to those skilled in the art, for example chromatography or crystallization and the individual enantiomers within each pair can be separated as described above. The present invention includes each diastereoisomer of such compounds and mixtures thereof.

As used herein, “a,” an” and “the” include singular and plural referents unless the context clearly dictates otherwise. Thus, for example, reference to “an active agent” or “a pharmacologically active agent” includes a single active agent as well a two or more different active agents in combination, reference to “a carrier” includes mixtures of two or more carriers as well as a single carrier, and the like.

The active compounds disclosed can, as noted above, be prepared in the form of their pharmaceutically acceptable salts. Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; (b) salts formed from elemental anions such as chlorine, bromine, and iodine, and (c) salts derived from bases, such as ammonium salts, alkali metal salts such as those of sodium and potassium, alkaline earth metal salts such as those of calcium and magnesium, and salts with organic bases such as dicyclohexylamine, N-methyl-D-glucamine, isopropylamine, trimethylamine, 2-ethylamino ethanol, histidine, procaine, and the like.

The active compounds disclosed can, as noted above, be prepared in the form of their hydrates, such as hemihydrate, monohydrate, dihydrate, trihydrate, tetrahydrate and the like.

This invention is also intended to encompass pro-drugs of the HDAC inhibitors disclosed herein. A prodrug of any of the compounds can be made using well known pharmacological techniques.

This invention, in addition to the above listed compounds, is intended to encompass the use of homologs and analogs of such compounds. In this context, homologs are molecules having substantial structural similarities to the above-described compounds and analogs are molecules having substantial biological similarities regardless of structural similarities.

Radiation Therapy

Radiation therapies which are suitable for use in the combination treatments described herein, include the use of a) external beam radiation; and b) a radiopharmaceutical agent which comprises a radiation-emitting radioisotope.

External Beam Radiation

External beam radiation therapy for the treatment of cancer uses a radiation source that is external to the patient, typically either a radioisotope, such as ⁶⁰Co, ¹³⁷Cs, or a high energy x-ray source, such as a linear accelerator. The external source produces a collimated beam directed into the patient to the tumor site. External-source radiation therapy avoids some of the problems of internal-source radiation therapy, but it undesirably and necessarily irradiates a significant volume of non-tumorous or healthy tissue in the path of the radiation beam along with the tumorous tissue.

The adverse effect of irradiating of healthy tissue can be reduced, while maintaining a given dose of radiation in the tumorous tissue, by projecting the external radiation beam into the patient at a variety of “gantry” angles with the beams converging on the tumor site. The particular volume elements of healthy tissue, along the path of the radiation beam, change, reducing the total dose to each such element of healthy tissue during the entire treatment.

The irradiation of healthy tissue also can be reduced by tightly collimating the radiation beam to the general cross section of the tumor taken perpendicular to the axis of the radiation beam. Numerous systems exist for producing such a circumferential collimation, some of which use multiple sliding shutters which, piecewise, can generate a radio-opaque mask of arbitrary outline.

Radiopharmaceutical Agents

A “radiopharmaceutical agent”, as defined herein, refers to a pharmaceutical agent which contains at least one radiation-emitting radioisotope. Radiopharmaceutical agents are routinely used in nuclear medicine for the diagnosis and/or therapy of various diseases. The radiolabelled pharmaceutical agent, for example, a radiolabelled antibody, contains a radioisotope (RI) which serves as the radiation source. As contemplated herein, the term “radioisotope” includes metallic and non-metallic radioisotopes. The radioisotope is chosen based on the medical application of the radiolabeled pharmaceutical agents. When the radioisotope is a metallic radioisotope, a chelator is typically employed to bind the metallic radioisotope to the rest of the molecule. When the radioisotope is a non-metallic radioisotope, the non-metallic radioisotope is typically linked directly, or via a linker, to the rest of the molecule.

As used herein, a “metallic radioisotope” is any suitable metallic radioisotope useful in a therapeutic or diagnostic procedure in vivo or in vitro. Suitable metallic radioisotopes include, but are not limited to: Actinium-225, Antimony-124, Antimony-125, Arsenic-74, Barium-103, Barium-140, Beryllium-7, Bismuth-206, Bismuth-207, Bismuth212, Bismuth213, Cadmium-109, Cadmium-115m, Calcium-45, Cerium-139, Cerium-141, Cerium-144, Cesium-137, Chromium-51, Cobalt-55, Cobalt-56, Cobalt-57, Cobalt-58, Cobalt-60, Cobalt-64, Copper-60, Copper-62, Copper-64, Copper-67, Erbium-169, Europium-152, Gallium-64, Gallium-67, Gallium-68, Gadolinium153, Gadolinium-157 Gold-195, Gold-199, Hafnium-175, Hafnium-175-181, Holmium-166, Indium-110, Indium-111, Iridium-192, Iron 55, Iron-59, Krypton85, Lead-203, Lead-210, Lutetium-177, Manganese-54, Mercury-197, Mercury203, Molybdenum-99, Neodymium-147, Neptunium-237, Nickel-63, Niobium95, Osmium-185+191, Palladium-103, Palladium-109, Platinum-195m, Praseodymium-143, Promethium-147, Promethium-149, Protactinium-233, Radium-226, Rhenium-186, Rhenium-188, Rubidium-86, Ruthenium-97, Ruthenium-103, Ruthenium-105, Ruthenium-106, Samarium-153, Scandium-44, Scandium-46, Scandium-47, Selenium-75, Silver-10m, Silver-111, Sodium-22, Strontium-85, Strontium-89, Strontium-90, Sulfur-35, Tantalum-182, Technetium-99m, Tellurium-125, Tellurium-132, Thallium-204, Thorium-228, Thorium-232, Thallium-170, Tin-113, Tin-114, Tin-117m, Titanium-44, Tungsten-185, Vanadium-48, Vanadium-49, Ytterbium-169, Yttrium-86, Yttrium-88, Yttrium-90, Yttrium-91, Zinc-65, Zirconium-89, and Zirconium-95.

As used herein, a “non-metallic radioisotope” is any suitable nonmetallic radioisotope (non-metallic radioisotope) useful in a therapeutic or diagnostic procedure in vivo or in vitro. Suitable non-metallic radioisotopes include, but are not limited to: Iodine-131, Iodine-125, Iodine-123, Phosphorus-32, Astatine-211, Fluorine-18, Carbon-11, Oxygen-15, Bromine-76, and Nitrogen-13.

Identifying the most appropriate isotope for radiotherapy requires weighing a variety of factors. These include tumor uptake and retention, blood clearance, rate of radiation delivery, half-life and specific activity of the radioisotope, and the feasibility of large-scale production of the radioisotope in an economical fashion. The key point for a therapeutic radiopharmaceutical is to deliver the requisite amount of radiation dose to the tumor cells and to achieve a cytotoxic or tumoricidal effect while not causing unmanageable side-effects.

It is preferred that the physical half-life of the therapeutic radioisotope be similar to the biological half-life of the radiopharmaceutical at the tumor site. For example, if the half-life of the radioisotope is too short, much of the decay will have occurred before the radiopharmaceutical has reached maximum target/background ratio. On the other hand, too long a half-life would cause unnecessary radiation dose to normal tissues. Ideally, the radioisotope should have a long enough half-life to attain a minimum dose rate and to irradiate all the cells during the most radiation sensitive phases of the cell cycle. In addition, the half-life of a radioisotope has to be long enough to allow adequate time for manufacturing, release, and transportation.

Other practical considerations in selecting a radioisotope for a given application in tumor therapy are availability and quality. The purity has to be sufficient and reproducible, as trace amounts of impurities can affect the radiolabeling and radiochemical purity of the radiopharmaceutical.

The target receptor sites in tumors are typically limited in number. As such it is preferred that the radioisotope have high specific activity. The specific activity depends primarily on the production method. Trace metal contaminants must be minimized as they often compete with the radioisotope for the chelator and their metal complexes compete for receptor binding with the radiolabeled chelated agent.

The type of radiation that is suitable for use in the methods of the present invention can vary. For example, radiation can be electromagnetic or particulate in nature. Electromagnetic radiation useful in the practice of this invention includes, but is not limited to, x-rays and gamma rays. Particulate radiation useful in the practice of this invention includes, but is not limited to, electron beams (beta particles), protons beams, neutron beams, alpha particles, and negative pi mesons. The radiation can be delivered using conventional radiological treatment apparatus and methods, and by intraoperative and stereotactic methods. Additional discussion regarding radiation treatments suitable for use in the practice of this invention can be found throughout Steven A. Leibel et al., Textbook of Radiation Oncology (1998) (publ. W. B. Saunders Company), and particularly in Chapters 13 and 14. Radiation can also be delivered by other methods such as targeted delivery, for example by radioactive “seeds,” or by systemic delivery of targeted radioactive conjugates. J. Padawer et al., Combined Treatment with Radioestradiol lucanthone in Mouse C3HBA Mammary Adenocarcinoma and with Estradiol lucanthone in an Estrogen Bioassay, Int. J. Radiat. Oncol. Biol. Phys. 7:347-357 (1981). Other radiation delivery methods can be used in the practice of this invention.

For tumor therapy, both α and β-particle emitters have been investigated. Alpha particles are particularly good cytotoxic agents because they dissipate a large amount of energy within one or two cell diameters. The β-particle emitters have relatively long penetration range (2-12 mm in the tissue) depending on the energy level. The long-range penetration is particularly important for solid tumors that have heterogeneous blood flow and/or receptor expression. The β-particle emitters yield a more homogeneous dose distribution even when they are heterogeneously distributed within the target tissue.

Modes and Doses of Administration

The methods of the present invention comprise administering to a patient in need thereof a first amount of a histone deacetylase inhibitor in a first treatment procedure, and a second amount or dose of radiation in a second treatment procedure. The first and second amounts together comprise a therapeutically effective amount.

“Patient” as that term is used herein, refers to the recipient of the treatment. Mammalian and non-mammalian patients are included. In a specific embodiment, the patient is a mammal, such as a human, canine, murine, feline, bovine, ovine, swine or caprine. In a particular embodiment, the patient is a human.

Administration of HDAC Inhibitor

The HDAC inhibitors of the invention can be administered in such oral forms as tablets, capsules (each of which includes sustained release or timed release formulations), pills, powders, granules, elixers, tinctures, suspensions, syrups, and emulsions. Likewise, the HDAC inhibitors can be administered in intravenous (bolus or infusion), intraperitoneal, subcutaneous, or intramuscular form, all using forms well known to those of ordinary skill in the pharmaceutical arts.

The HDAC inhibitors can be administered in the form of a depot injection or implant preparation which can be formulated in such a manner as to permit a sustained release of the active ingredient. The active ingredient can be compressed into pellets or small cylinders and implanted subcutaneously or intramuscularly as depot injections or implants. Implants can employ inert materials such as biodegradable polymers or synthetic silicones, for example, Silastic, silicone rubber or other polymers manufactured by the Dow-Corning Corporation.

The HDAC inhibitor can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The HDAC inhibitors can also be delivered by the use of monoclonal antibodies as individual carriers to which the compound molecules are coupled.

The HDAC inhibitors can also be prepared with soluble polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the HDAC inhibitors can be prepared with biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

The dosage regimen utilizing the HDAC inhibitors can be selected in accordance with a variety of factors including type, species, age, weight, sex and the type of cancer being treated; the severity (i.e., stage) of the cancer to be treated; the route of administration; the renal and hepatic function of the patient; and the particular compound or salt thereof employed. An ordinarily skilled physician or veterinarian can readily determine and prescribe the effective amount of the drug required to treat, for example, to prevent, inhibit (fully or partially) or arrest the progress of the disease.

Oral dosages of HDAC inhibitors, when used to treat the desired cancer, can range between about 2 mg to about 2000 mg per day, such as from about 20 mg to about 2000 mg per day, such as from about 200 mg to about 2000 mg per day. For example, oral dosages can be about 2, about 20, about 200, about 400, about 800, about 1200, about 1600 or about 2000 mg per day. It is understood that the total amount per day can be administered in a single dose or can be administered in multiple dosing such as twice, three or four times per day.

For example, a patient can receive between about 2 mg/day to about 2000 mg/day, for example, from about 20-2000 mg/day, such as from about 200 to about 2000 mg/day, for example from about 400 mg/day to about 1200 mg/day. A suitably prepared medicament for once a day administration can thus contain between about 2 mg and about 2000 mg, such as from about 20 mg to about 2000 mg, such as from about 200 mg to about 1200 mg, such as from about 400 mg/day to about 1200 mg/day. The HDAC inhibitors can be administered in a single dose or in divided doses of two, three, or four times daily. For administration twice a day, a suitably prepared medicament would therefore contain half of the needed daily dose.

Intravenously or subcutaneously, the patient would receive the HDAC inhibitor in quantities sufficient to deliver between about 3-1500 mg/m² per day, for example, about 3, 30, 60, 90, 180, 300, 600, 900, 1200 or 1500 mg/m² per day. Such quantities can be administered in a number of suitable ways, e.g. large volumes of low concentrations of HDAC inhibitor during one extended period of time or several times a day. The quantities can be administered for one or more consecutive days, intermittent days or a combination thereof per week (7 day period). Alternatively, low volumes of high concentrations of HDAC inhibitor during a short period of time, e.g. once a day for one or more days either consecutively, intermittently or a combination thereof per week (7 day period). For example, a dose of 300 mg/m² per day can be administered for 5 consecutive days for a total of 1500 mg/m² per treatment. In another dosing regimen, the number of consecutive days can also be 5, with treatment lasting for 2 or 3 consecutive weeks for a total of 3000 mg/m² and 4500 mg/r² total treatment.

Typically, an intravenous formulation can be prepared which contains a concentration of HDAC inhibitor of between about 1.0 mg/mL to about 10 mg/mL, e.g. 2.0 mg/mL, 3.0 mg/mL, 4.0 mg/mL, 5.0 mg/mL, 6.0 mg/mL, 7.0 mg/mL, 8.0 mg/mL, 9.0 mg/mL and 10 mg/mL and administered in amounts to achieve the doses described above. In one example, a sufficient volume of intravenous formulation can be administered to a patient in a day such that the total dose for the day is between about 300 and about 1500 mg/m².

Glucuronic acid, L-lactic acid, acetic acid, citric acid or any pharmaceutically acceptable acid/conjugate base with reasonable buffering capacity in the pH range acceptable for intravenous administration of the HDAC inhibitor can be used as buffers. Sodium chloride solution wherein the pH has been adjusted to the desired range with either acid or base, for example, hydrochloric acid or sodium hydroxide, can also be employed. Typically, a pH range for the intravenous formulation can be in the range of from about 5 to about 12. A preferred pH range for intravenous formulation wherein the HDAC inhibitor has a hydroxamic acid moiety, can be about 9 to about 12. Consideration should be given to the solubility and chemical compatibility of the HDAC inhibitor in choosing an appropriate excipient.

Subcutaneous formulations, preferably prepared according to procedures well known in the art at a pH in the range between about 5 and about 12, also include suitable buffers and isotonicity agents. They can be formulated to deliver a daily dose of HDAC inhibitor in one or more daily subcutaneous administrations, e.g., one, two or three times each day. The choice of appropriate buffer and pH of a formulation, depending on solubility of the HDAC inhibitor to be administered, is readily made by a person having ordinary skill in the art. Sodium chloride solution wherein the pH has been adjusted to the desired range with either acid or base, for example, hydrochloric acid or sodium hydroxide, can also be employed in the subcutaneous formulation. Typically, a pH range for the subcutaneous formulation can be in the range of from about 5 to about 12. A preferred pH range for subcutaneous formulation wherein the HDAC inhibitor has a hydroxamic acid moiety, can be about 9 to about 12. Consideration should be given to the solubility and chemical compatibility of the HDAC inhibitor in choosing an appropriate excipient.

The HDAC inhibitors can also be administered in intranasal form via topical use of suitable intranasal vehicles, or via transdermal routes, using those forms of transdermal skin patches well known to those of ordinary skill in that art. To be administered in the form of a transdermal delivery system, the dosage administration will, or course, be continuous rather than intermittent throughout the dosage regime.

The HDAC inhibitors can be administered as active ingredients in admixture with suitable pharmaceutical diluents, excipients or carriers (collectively referred to herein as “carrier” materials) suitably selected with respect to the intended form of administration, that is, oral tablets, capsules, elixers, syrups and the like, and consistent with conventional pharmaceutical practices.

For instance, for oral administration in the form of a tablet or capsule, the HDAC inhibitor can be combined with an oral, non-toxic, pharmaceutically acceptable, inert carrier such as lactose, starch, sucrose, glucose, methyl cellulose, microcrystalline cellulose, sodium croscarmellose, magnesium stearate, dicalcium phosphate, calcium sulfate, mannitol, sorbitol and the like or a combination thereof, for oral administration in liquid form, the oral drug components can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, microcrystalline cellulose, sodium croscarmellose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch methyl cellulose, agar, bentonite, xanthan gum and the like.

Suitable pharmaceutically acceptable salts of the histone deacetylase compounds described herein and suitable for use in the method of the invention, are conventional non-toxic salts and can include a salt with a base or an acid addition salt such as a salt with an inorganic base, for example, an alkali metal salt (e.g. lithium salt, sodium salt, potassium salt, etc.), an alkaline earth metal salt (e.g. calcium salt, magnesium salt, etc.), an ammonium salt; a salt with an organic base, for example, an organic amine salt (e.g. triethylamine salt, pyridine salt, picoline salt, ethanolamine salt, triethanolamine salt, dicyclohexylamine salt, N,N′-dibenzylethylenediamine salt, etc.) etc.; an inorganic acid addition salt (e.g. hydrochloride, hydrobromide, sulfate, phosphate, etc.); an organic carboxylic or sulfonic acid addition salt (e.g. formate, acetate, trifluoroacetate, maleate, tartrate, methanesulfonate, benzenesulfonate, p-toluenesulfonate, etc.); a salt with a basic or acidic amino acid (e.g. arginine, aspartic acid, glutamic acid, etc.) and the like.

The histone deacetylase inhibitors and radiation can also be used in a method of treating cancer in a cell comprising contacting the cell with a first amount of a compound capable of inhibiting a histone deacetylase or a salt thereof and contacting the cell with a second amount of radiation therapy, to prevent, inhibit (fully or partially) or arrest the progress of the cancer. The cell can be a transgenic cell. In another embodiment the cell can be in a patient, such as a mammal, for example a human.

In certain embodiments, the first amount to treat cancer in a cell is a contact concentration of HDAC inhibitor from about 1 pM to about 50 μM such as, from about 1 pM to about 5 μM., for example, from about 1 pM to about 500 nM, such as from about 1 pM to about 50 mM, for example, 1 pM to about 500 pM. In a particular embodiment, the concentration is less than about 5.0 μM. In another embodiment, the concentration is about 500 nM.

Administration of External Beam Radiation

For administration of external beam radiation, the amount can be at least about 1 Gray (Gy) fractions at least once every other day to a treatment volume. In a particular embodiment, the radiation is administered in at least about 2 Gray (Gy) fractions at least once per day to a treatment volume. In another particular embodiment, the radiation is administered in at least about 2 Gray (Gy) fractions at least once per day to a treatment volume for five consecutive days per week. In another particular embodiment, radiation is administered in 10 Gy fractions every other day, three times per week to a treatment volume. In another particular embodiment, a total of at least about 20 Gy is administered to a patient in need thereof. In another particular embodiment, at least about 30 Gy is administered to a patient in need thereof. In another particular embodiment, at least about 40 Gy is administered to a patient in need thereof.

Typically, the patient receives external beam therapy four or five times a week. An entire course of treatment usually lasts from one to seven weeks depending on the type of cancer and the goal of treatment. For example, a patient can receive a dose of 2 Gy/day over 30 days.

Administration Radiopharmaceutical Agent

There are a number of methods for administration of a radiopharmaceutical agent. For example, the radiopharmaceutical agent can be administered by targeted delivery or by systemic delivery of targeted radioactive conjugates, such as a radiolabeled antibody, a radiolabeled peptide and a liposome delivery system.

In one particular embodiment of targeted delivery, the radiolabelled pharmaceutical agent can be a radiolabelled antibody. See, for example, Ballangrud A. M., et al. Cancer Res., 2001; 61:2008-2014 and Goldenber, D. M. J. Nucl. Med., 2002; 43(5):693-713, the contents of which are incorporated by reference herein.

In another particular embodiment of targeted delivery, the radiopharmaceutical agent can be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines. See, for example, Emfietzoglou D, Kostarelos K, Sgouros G. An analytical dosimetry study for the use of radionuclide-liposome conjugates in internal radiotherapy. J Nucl Med 2001; 42:499-504, the contents of which are incorporated by reference herein.

In yet another particular embodiment of targeted delivery, the radiolabled pharmaceutical agent can be a radiolabeled peptide. See, for example, Weiner R E, Thakur M L. Radiolabeled peptides in the diagnosis and therapy of oncological diseases. Appl Radiat Isot 2002 Nov;57(5):749-63, the contents of which are incorporated by reference herein.

In addition to targeted delivery, Brachytherapy can be used to deliver the radiopharmaceutical agent to the target site. Brachytherapy is a technique that puts the radiation sources as close as possible to the tumor site. Often the source is inserted directly into the tumor. The radioactive sources can be in the form of wires, seeds or rods. Generally, cesium, iridium or iodine are used.

There a two types of brachytherapy: intercavitary treatment and interstitial treatment. In intracavitary treatment, containers that hold radioactive sources are put in or near the tumor. The sources are put into the body cavities.

In interstitial treatment the radioactive sources alone are put into the tumor. These radioactive sources can stay in the patient permanently. Most often, the radioactive sources are removed from the patient after several days. The radioactive sources are in containers.

In addition, a radiopharmaceutical agent can be administered to a patient using any one of the modes of administration detailed hereinabove for the HDAC inhibitors.

The amount of radiation necessary can be determined by one of skill in the art based on known doses for a particular type of cancer. See, for example, Cancer Medicine 5^{th} ed., Edited by R. C. Bast et al., July 2000, B C Decker, the entire content of which is hereby incorporated by reference.

In a particular embodiment, the radiation can be administered in amount effective to cause the arrest or regression of the cancer of, when the radiation is administered with the HDAC inhibitor.

Combination Administration

The first treatment procedure, administration of a histone deacetylase inhibitor, can take place prior to the second treatment procedure, radiation, after the radiation treatment, at the same time as the radiation or a combination thereof. The first and second amounts can be combined prior to administration or administered at different sites but at the same time. For example, a total treatment period can be decided for the histone deacetylase inhibitor. The radiation can be administered prior to onset of treatment with the inhibitor or following treatment with the inhibitor. In addition, radiation treatment can be administered during the period of inhibitor administration but does not need to occur over the entire inhibitor treatment period.

The following examples more fully illustrate the preferred embodiments of the invention. They should in no way be construed, however, as limiting the broad scope of the invention.

Experimental Methods

Materials and Methods

CELL CULTURE: The human prostate carcinoma cell line LNCaP (CRL 1740) was purchased from the ATCC (Manassas, Va.). Stock T-Flask cultures were propagated at 37° C., 95% relative humidity, and 5% CO₂ in RPMI 1640 (Invitrogen, Carlsbad, Calif.) supplemented with 10% fetal calf serum (Sigma, St. Louis, Mo.), 100 units/mL penicillin, and 100 mg/mL streptomycin (Gemini Bio-products, Woodland, Calif.). Cell concentrations were determined by counting trypsinized cells with a hemocytometer.

SPHEROID INITIATION: Tumor Cell Clusters or Spheroids were initiated according to the liquid overlay technique of Yuhas et al. See, Yuhas J. M. et al. Cancer Res., 37: 3639-3643, 1977. Details regarding LNCaP spheroid formation and characterization are described in Ballangrud A. M. et al. Clin. Cancer Res., 5: 3171 s-3176s, 1999. The entire content of the above references is hereby incorporated by reference.

Briefly, liquid overlay plates were prepared from 100 mm or 35 mm petri dishes (Becton Dickinson Labware, Franklin Lakes, N.J.) containing a thin layer of RPMI 1640 media solidified with 1% agar (Difco, Detroit, Mich.). The medium was inoculated at 6.7×10⁴ cells/mL from trypsinized stock cultures. The resulting suspension was used to seed 100 mm plates with approximately 10⁶ cells. After an incubation of 5-7 days, spheroids of ˜200 μm diameter were selected under an inverted phase-contrast microscope (Axiophot 2; Carl Zeiss Ltd., Göttingen, Germany) fitted with an ocular scale using an Eppendorf pipette.

TREATMENT PROTOCOLS: After each treatment, spheroids were washed three times by suspension in fresh medium. Complete treatment consisted of either incubation with SAHA, irradiation, or exposure to both SAHA and radiation. A minimum of 12 spheroids was used for each condition in duplicate experiments.

For SAHA incubation, a 10 mM stock solution in DMSO was serially diluted in media to produce 1-5 μM SAHA and to generate final DMSO concentrations of <0.01%. 12-24 washed spheroids were placed in an agar-prepared 35 mm petri dish as described above and covered with sufficient SAHA containing media to cover the entire agar surface.

For external-beam irradiation, spheroids were exposed to an acute dose of 6 Gy external beam photon irradiation using a Cesium irradiator at a dose rate of 2.3 Gy/min (Cs-137 Model 68; J L Shepherd and Associates, Glendale, Calif.).

For alpha-emitting radiation, spheroids were exposed to a radioactivity concentration of (100 nCi/mL) of Ac225-HuM 195 alpha emitting radiation for 24 hours. Ac225-HuM 195 is a recombinant humanized Anti-CD33 monoclonal antibody which has been radiolabelled with actinium225. The antibody was obtained from the laboratory of David Scheinberg, M.D., Ph.D., Memorial Sloan-Kettering Cancer Center.

Following complete treatment, washed spheroids were placed in separate agar-prepared wells of a 24 well plate. Untreated spheroids were washed and separated immediately after initial selection. The medium in each well was replaced, and volume measurements preformed, twice per week. Using the inverted microscope and ocular scale described previously, the major and minor diameter, dₘₐₓ and dₘᵢₙ respectively, were determined and spheroid volume calculated as V=(1/6)·π·dₘₐₓ·dₘᵢₙ². Volume monitoring was stopped once a spheroid had exceeded the field of view of the microscope or had fragmented into individual cells or multiple smaller cell clusters. At the end of each experiment, spheroids that did not regrow were scored for viability using an outgrowth assay—cells or spheroid fragments from wells containing spheroids that did not regrow were collected and placed in individual wells of a separate, agar-free (adherent) 24-well plate, incubated for 2 weeks and then scored for colonies.

IMMUNOHISTOCHEMISTRY: Proliferation or apoptosis of tumor cells within spheroids was assessed by Ki67 or TdT-mediated dUTP-biotin nick end labeling (TUNEL) staining, respectively. At 0, 6, 24, or 48 h post treatment spheroids were washed in cold media, fixed for 4 hr in 4% paraformaldehyde, and placed in paraffin blocks. Serial 5-μm sections of the blocks were cut using a microtome and mounted on poly-L-lysine-coated slides that were fixed in ice cold acetone for 10 min.

Ki67 staining was performed using a monoclonal mouse antibody directed against Ki67 and MOM kit (Vector Labs, Burlingame, Calif.).

Apoptotic cells were stained using TUNEL modified from Gavrieli et al. (J Cell Biol. 119: 493-501., 1992). A final, 2 min incubation in Hematoxylin was used to counterstain sections. Untreated spheroids were used as controls; positive controls were created using DNase I (Boehringer, Ingelheim, Germany). Images were captured digitally from an inverted phase-contrast microscope using a coupled Pixera Professional Camera and associated software (Pixera Visual Communication Suite, Pixera, Los Gatos, Calif.). Images were scored for positive staining as percentage of reactive cells within the spheroid section.

STATISTICAL ANALYSIS: To assess synergy between SAHA and radiation, the area under the tumor volume curve (AUC) was measured for each spheroid. Synergistic inhibition of tumor growth is defined as the combination treatment group producing on average a smaller log AUC than predicted by the additive model that includes each treatment group separately. We describe this relationship through the inequality:

avg(V|S=5M, R=6 Gy)<C+{avg(V|S=5 μM, R=0)−C}+{avg(V|S=0, R=6 Gy)−C}

where V is the log AUC, S=5 μM and R=6 Gy represent the doses of SAHA and radiation used in the experiment, and C is the average log AUC in the control group [C=avg(V|S=0, R=0)]. To test for synergy, we computed 2000 bootstrap replicates of the average log AUC, for each of the four groups, and computed the proportion of replicates where the inequality was not obtained. This proportion is termed the achieved significance level (p-value). A small achieved significance level is an indication that synergistic inhibition of tumor growth has occurred due to the combination treatment.

A two-tailed T test was used to test for significant differences in the percentage of positively stained cells.

Results:

### EXAMPLE 1

Effect of SAHA on Spheroid Growth

Studies were carried out in spheroids whose response to chemotherapeutics and radiation has been shown to better approximate the response seen in tumors, in vivo (Stuschke, M. et al. Int J Radiat Oncol Biol Phys. 24: 119-26, 1992; Santini, M. T. et al. Int J Radiat Biol. 75: 787-99, 1999; Dertinger, H. et al. Radiat Environ Biophys. 19: 101-7, 1981).

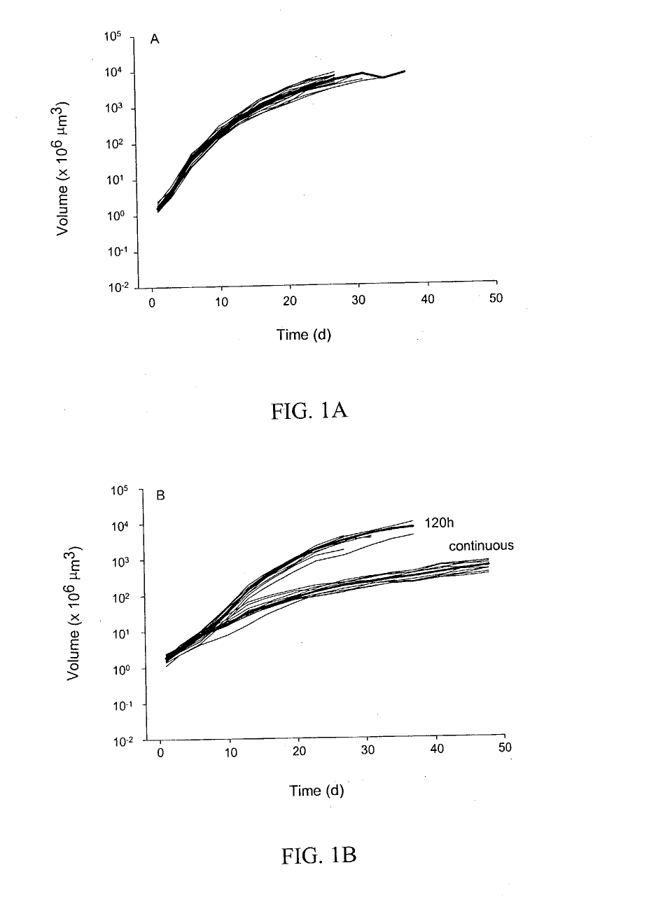

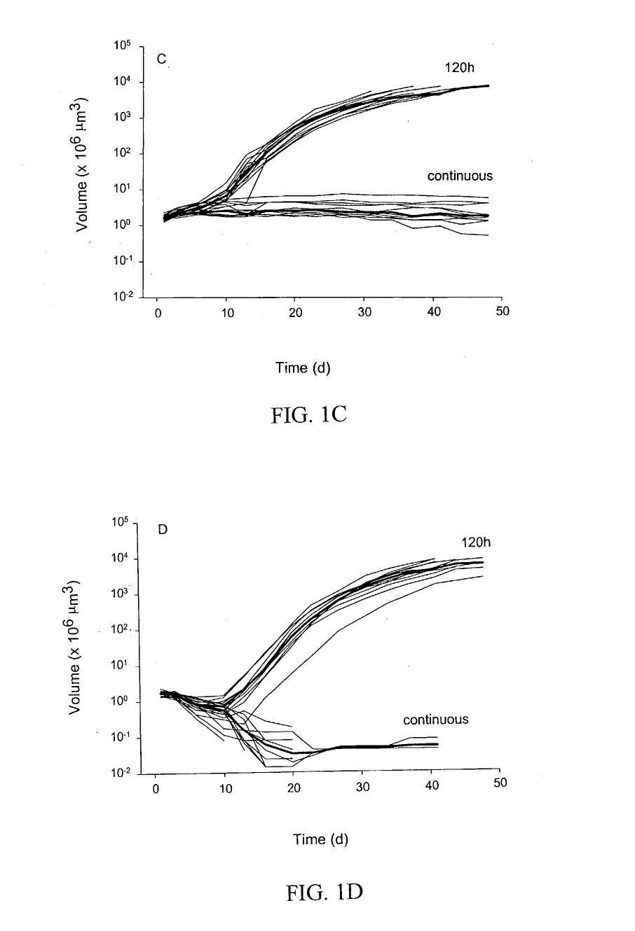

The effect of SAHA on spheroid growth was examined by incubating spheroids with 0, 1.25, 2.5 and 5 μM SAHA either for 120 h or continuously (FIGS 1A-D). Spheroid growth was monitored for at least 40 days following incubation with SAHA for 120 h or for the 40-day continuous treatment. At a concentration of 1.25 μM SAHA, spheroid growth was delayed but not arrested for both the 120-hour and continuous exposure conditions. At 2.5 μM, complete growth arrest was observed over the 120 h incubation period. Growth inhibition persisted for another 4 to 5 days after the end of drug exposure. This delay in recovery was then followed by exponential growth followed by a plateau (i.e., Gompertzian growth (Bassukas, I. D. Cancer Res. 54: 4385-92., 19949)) similar to that obtained with untreated spheroids. Five days following a 120 hour incubation with 5 μM SAHA, a 2.4-fold median volume reduction was seen after which spheroid growth returned to Gompertzian kinetics. The time required for spheroids to reach a volume 1000-fold greater (Approx. 10 volume doubling times) than the starting volume after 0 (no SAHA exposure), 1.25, 2.5 and 5 μM-SAHA (5-day incubation) was 16, 20, 23 and 29 days, respectively; yielding 4, 7 and 13 day growth delays, respectively, for the SAHA-treated spheroids.

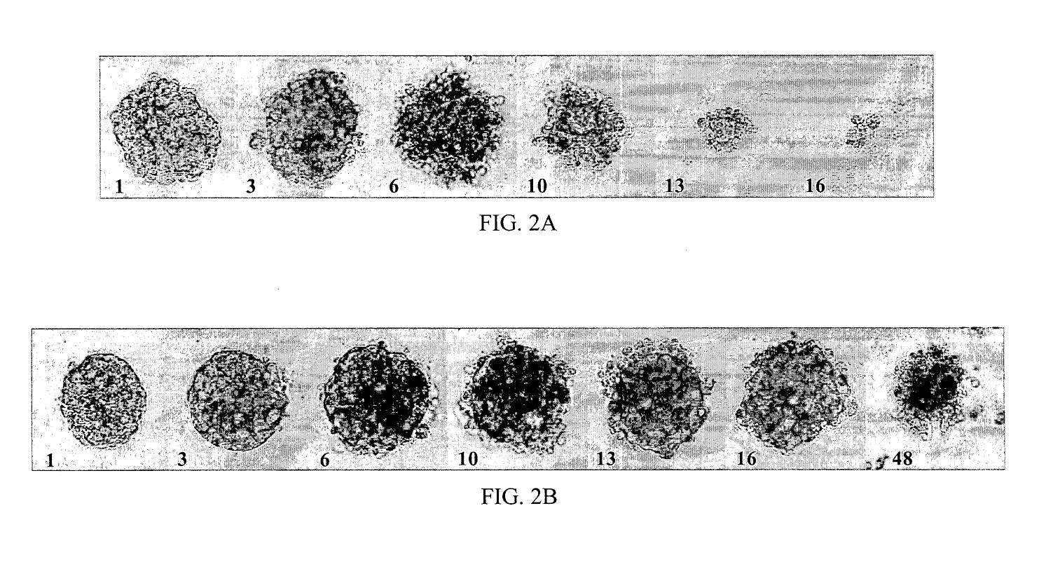

Continuous exposure of spheroids at 2.5 μM resulted in complete growth suppression; at 5 μM, a rapid loss in spheroid volume was observed with the majority of spheroids disaggregating by day 20. Typical morphology of arrested or disaggregating spheroids is shown in FIG. 2A (5 μM) and FIG. 2B (2.5 μM).

To evaluate the activity of SAHA as an anti-tumor cell agent, it is instructive to compare results obtained using spheroids with monolayer culture experiments. In LNCaP monolayer cell culture, 2.5 μM SAHA causes complete growth suppression with minimal to no cell kill over a 4-day period and 5 μM SAHA causes progressive cell kill starting after 48 hours of SAHA incubation (Butler, L. M. et al. Cancer Res. 60. 5165-70., 2000).

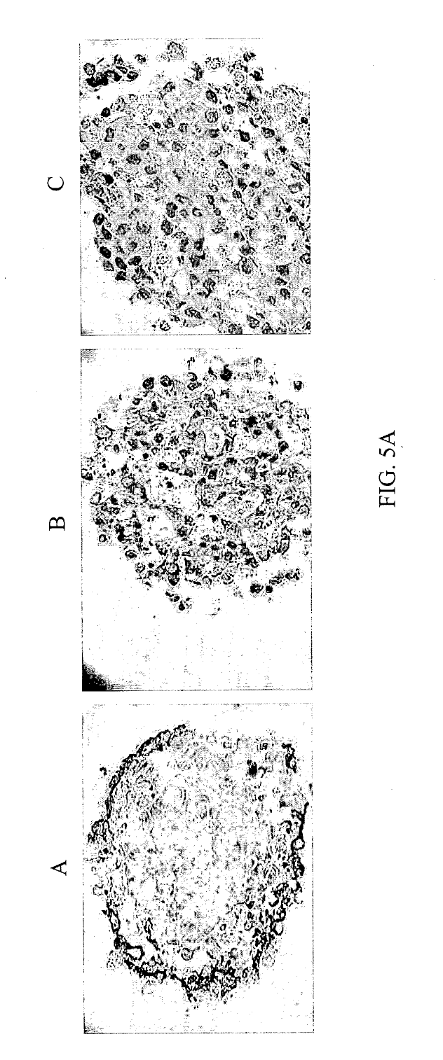

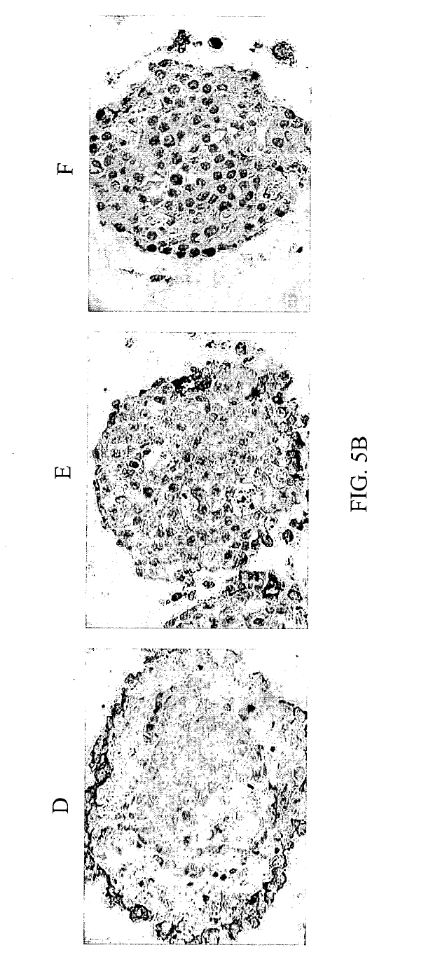

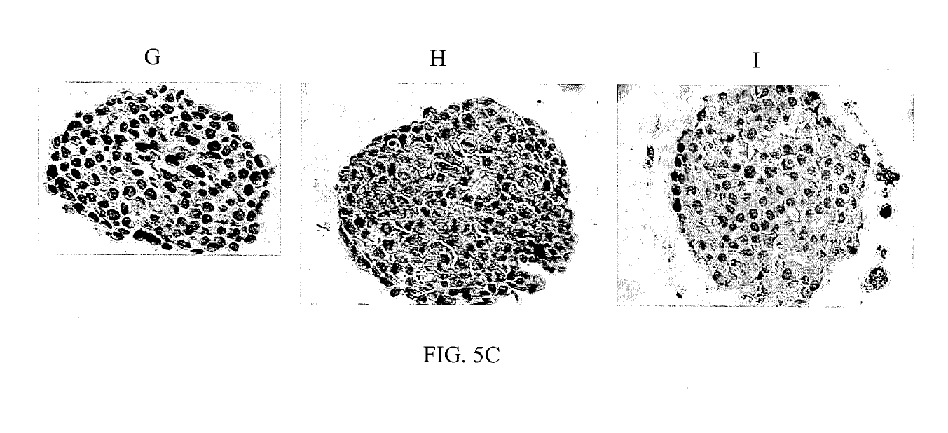

In these experiments, the volume response of LNCaP spheroids to these concentrations was generally consistent with the monolayer culture results (FIGS. 1A-D). The images (FIGS. 2A-B), however, revealed that the time-scale and etiology for these effects was different from that seen in monolayer culture. At 5 μM, complete spheroid disruption did not occur until after 13 to 16 days of incubation with SAHA; and the apparent growth inhibition at 2.5 μM appeared to arise primarily due to the continuous loss of cells on the spheroid surface. As shown by TUNEL staining (see below and FIGS. 5A-C) and as suggested by the morphology and rapid elimination of cells in spheroids (FIG. 2A), cell death following exposure to SAHA is predominantly by apoptosis.

### EXAMPLE 2

Effect of SAHA and External Beam Radiation on Spheroid Growth

The dose-response of LNCaP spheroids to external beam, low LET, high dose-rate irradiation has been reported previously (Ballangrud, A. M. et al. Cancer Res. 61: 2008-14., 2001; Enmon, R. M., et al. Cancer Res, submitted). Based on these data, absorbed doses of 3 and 6 Gy were selected in the combination studies since these doses of radiation alone, yielded growth curves that matched the untreated curve in shape but with delays of 4 to 10 days to reach 1000-fold the original spheroid volume. Based on the SAHA dose-response data (FIGS. 1A-D), a 96 h incubation with 5 μM SAHA was selected for the combination studies. Combination treatment was carried out by exposing spheroids to SAHA for 48 h, irradiating and then incubating for another 48 or 72 h prior to washing and monitoring for growth.

LNCaP cells grown as spheroids were used in this study. The following treatment regimen was used:

A: No treatment

B: Treatment with 5 μM SAHA for 96 hours.

C: Treatment with 6 Gy acute irradiation using a Cs-137 irradiator at a dose rate of 2.3 Gy/min (Cs-137 Model 68: J L Shepherd and Associated, Glendale, Calif.) Treatment was uniform across the spheroid and a low LET of 0.2 keV/μm was used.

D: Treatment with 5 μM SAHA for a total of 96 hours, with a 6 Gy acute irradiation using a Cs-137 irradiator (as describe above) following 48 of the 96 total hours of SAHA exposure.

Combination studies with 3 Gy (and 120 h SAHA) yielded modest SAHA-dose dependent delays in spheroid growth; at 5 μM concentration a 7-day delay was observed (data not shown).

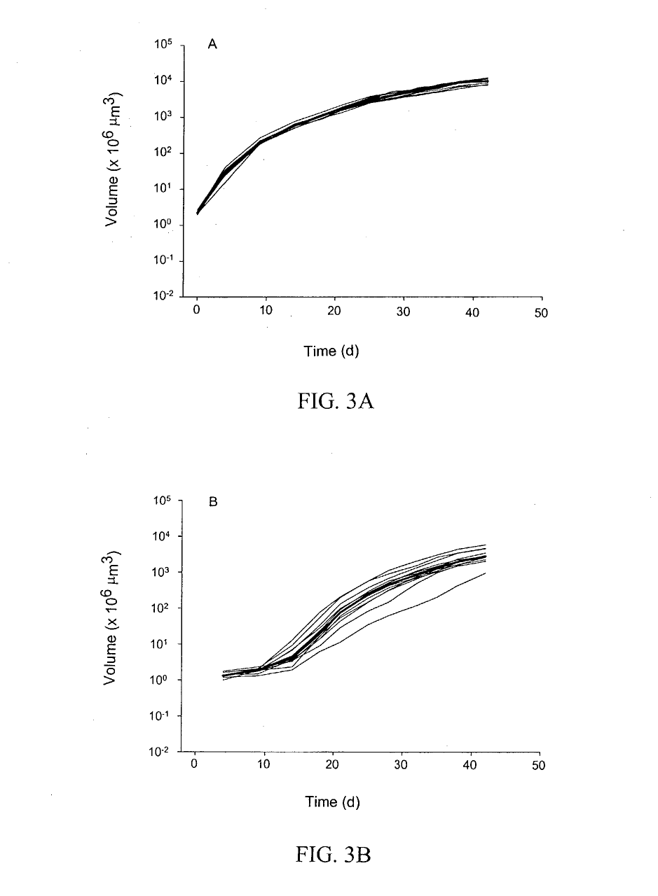

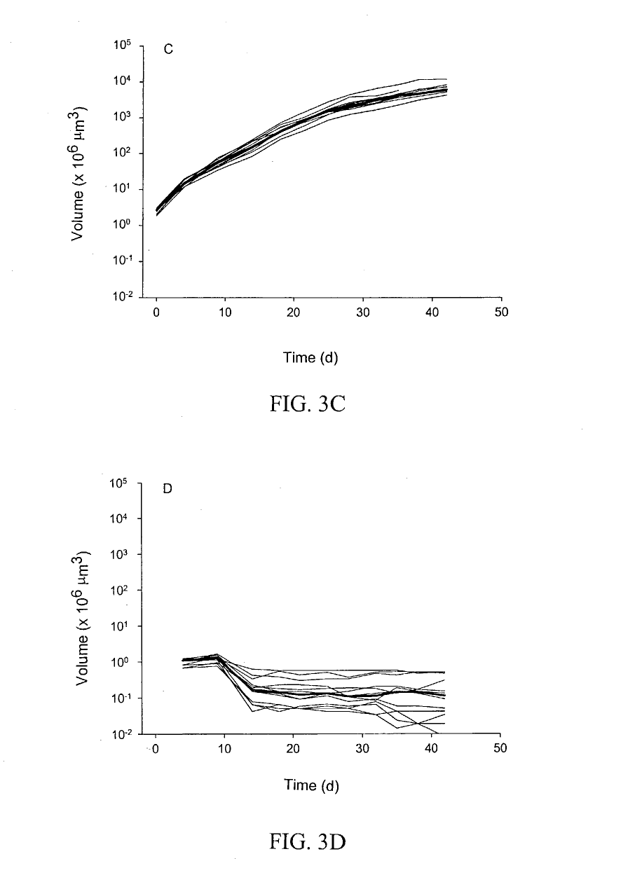

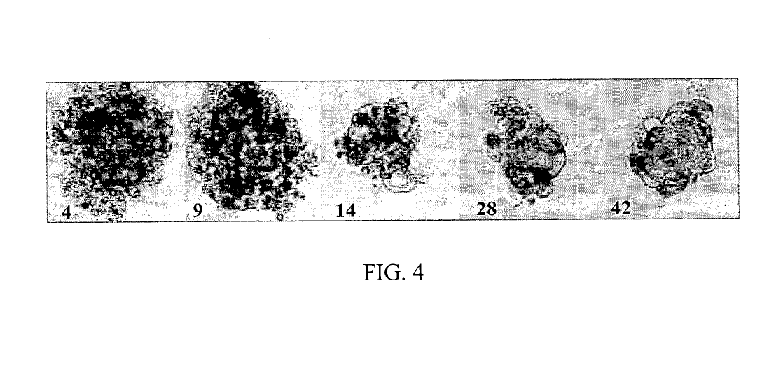

Combination studies with 6 Gy and a 96 h incubation with 5 μM SAHA caused complete growth inhibition with none of the 12 spheroids forming colonies in the outgrowth assay (FIG. 3D). In contrast, 6 Gy radiation alone (FIG. 3C) or a 96 h exposure to 5 μM SAHA alone (FIG. 3B) yielded 5 and 15-day delays, respectively, for a 1000-fold increase in original volume. Statistical analysis of these results indicated synergistic inhibition of tumor growth resulting from combination treatment (p<0.01). Typical spheroid morphology at different times after combination therapy is depicted in FIG. 4. Soon after the end of treatment (days 4 and 9), spheroids have an appearance that is similar to that seen with SAHA only treatment. At later times, spheroid morphology is altered considerably; the spheroids appear to be composed of a small number of swollen, possibly necrotic cells.

### EXAMPLE 3

Effect of SAHA and External Beam Radiation on Apoptosis

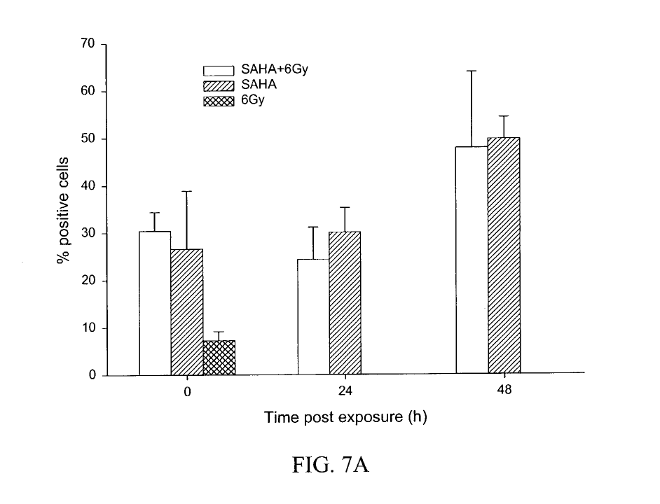

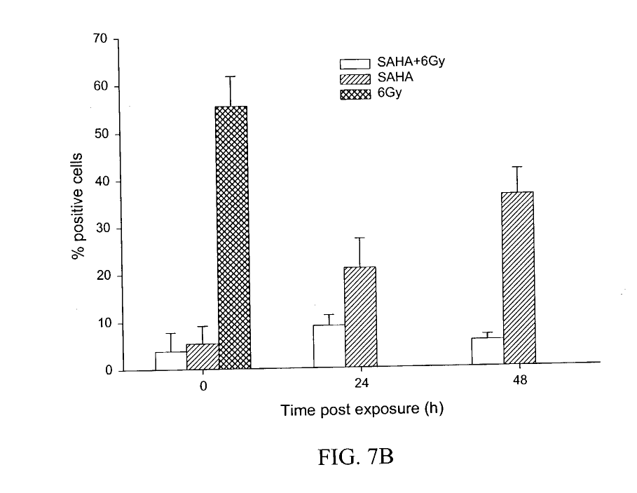

To examine whether SAHA increases radiation-induced apoptosis, TUNEL staining of spheroid sections at various times after the end of single or combination therapy was carried out. Immediately after the end of a 96 h SAHA incubation the majority of cells on the spheroid surface have undergone apoptosis and there is little evidence of apoptosis in the spheroid interior (FIG. 5A). This finding is also consistent with the morphological appearance of SAHA-treated spheroids at days 3 and 6 (FIG. 2A). By 48 h after the end of SAHA incubation, the apoptotic cells on the spheroid surface are not detected, presumably due to shedding, and apoptotic cells are found throughout the spheroid. Pockets of cellular debris are also evident within the interior. These are evident immediately after the end of SAHA incubation but become more prominent 6 and 24 hours later. TUNEL staining of spheroids treated with SAHA and radiation yielded an almost identical pattern suggesting that, although SAHA induces substantial apoptosis, the synergistic spheroid response seen with the combination cannot be explained by enhanced apoptosis (FIG. 7A).

### EXAMPLE 4

Effect of SAHA and External Beam Radiation on Proliferation

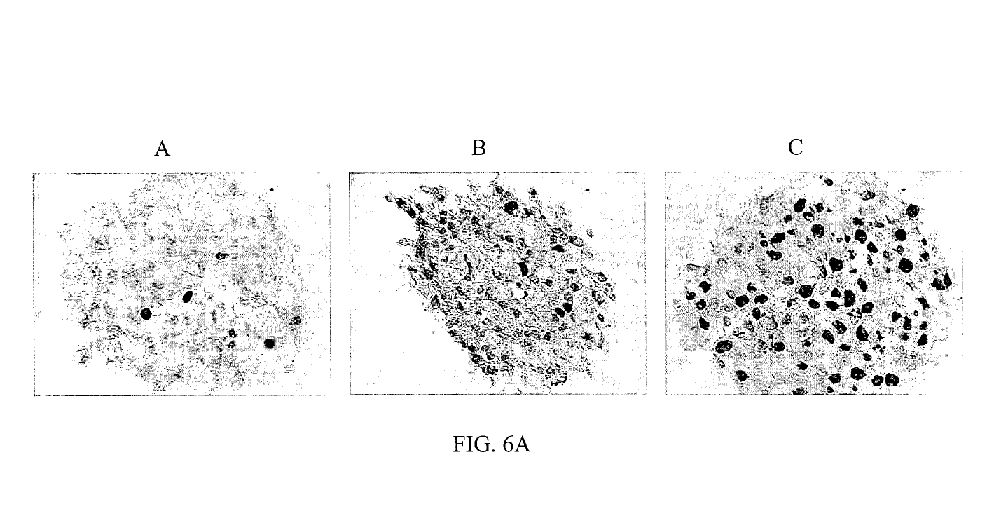

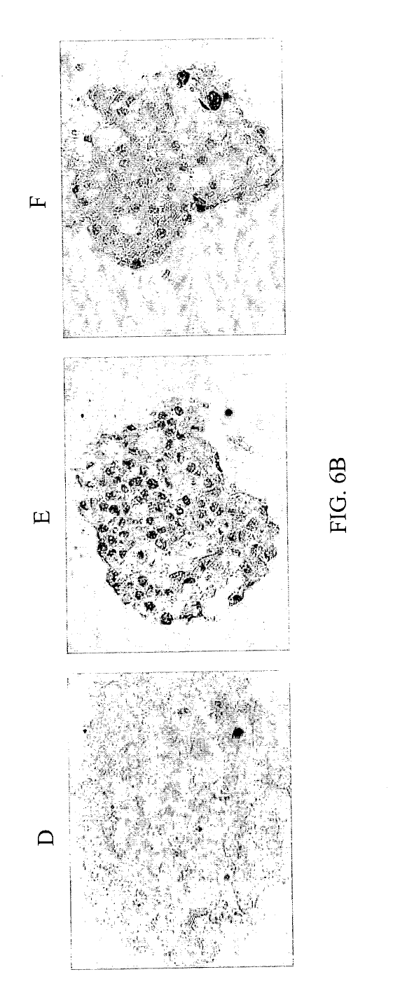

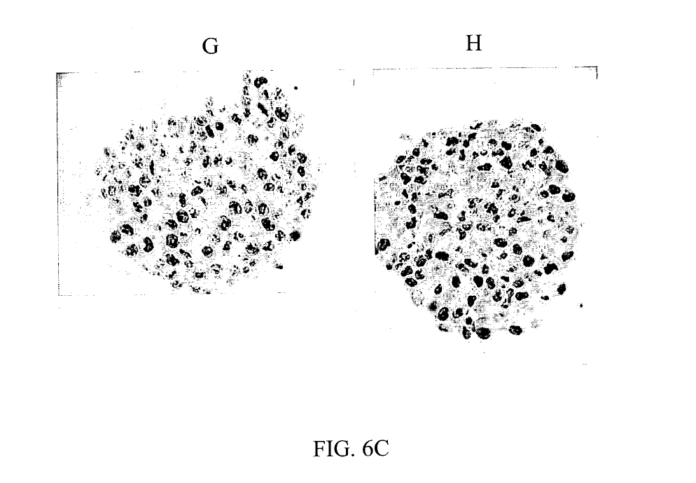

In contrast to the TUNEL staining results shown in Example 3, corresponding immunohistochemistry studies examining proliferative activity by Ki67 staining showed substantial differences in cellular proliferation for each treatment alone, versus the combination (FIGS. 6A-C and 7B). At the end of the 96 h incubation with SAHA, virtually all cells making up the spheroids have stopped cycling. This is consistent with the known cell-cycle inhibitory effects of SAHA. The inhibitory effects are short-lived and within 6 to 24 hours faint positive Ki67 staining can be seen. By 48 hours, many cells throughout the section show intense Ki67 staining in spheroids treated only with SAHA. No such staining is seen in spheroids treated with the combination (p<0.01).

### EXAMPLE 5

Effect of SAHA and Alpha Radiation on Spheroid Growth

To test the effect of a combination of SAHA and an alpha-particle emitting radioisotope, combination treatment was carried out by exposing spheroids for 24 hours to 100 nCi/mL of Ac-225 prior to exposure to SAHA for 96 h.

LNCaP cells grown as spheroids as above. The following treatment regimen was used:

A: No treatment

B: Treatment with 5 μM SAHA for 96 hours.

C: Treatment with 100 nCi/mL Ac225-HuM195 for 24 hours.

D: Treatment with 5 μM SAHA for a total of 96 hours, in combination with 100 nCi/mL Ac225-HuM 195 treatment for 24 hours prior to SAHA treatment.

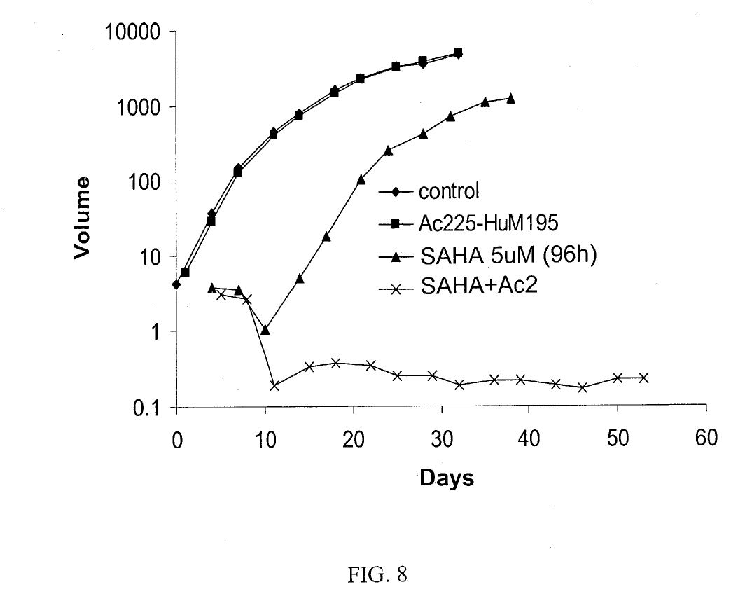

Combination studies with 24 hour exposure to Ac225-HuM195 followed by a 96 h incubation with 5 μM SAHA caused complete growth inhibition, which was maintained for a period of over 50 days (FIG. 8). In contrast, Ac225-HuM195 treatment alone did not cause growth inhibition of the spheroids, and a 96 h exposure to 5 μM SAHA alone yielded an initial 10 day delay, followed by spheroid growth to almost the levels of control untreated spheroid volume after a period of 30 days. The anti-CD33 antibody was chosen in these experiments since it is known not to bind to prostate cancer cells or spheroids, thereby allowing control of the incubation period and alpha-particle dose delivered by the Ac225 radionuclide. Use of an “irrelevant” antibody makes it easier to calculate the absorbed dose delivered to the spheroids since there is no retention of radioactivity beyond the incubation period. This is important in establishing a dose-response relationship and in ensuring that the observed synergistic effects are due primarily to the combination of SAHA and radiation rather than antibody mediated effects. In practice, a specific antibody that recognizes antigen sites on tumor cells can be used to deliver the radionuclide.

Summary of the Findings

The combination of radiation and SAHA yielded growth suppression that led to 10- to 100-thousand-fold differences in spheroid volumes relative to each modality alone (FIGS. 3A-D. The results of TUNEL staining for SAHA only- versus combination-treated spheroids suggests that the synergistic increase in efficacy does not appear to arise as a result of enhanced apoptosis (FIG. 5A versus FIG. 5B and FIG. 7A). This observation is consistent with the morphological characteristics of spheroids immediately after the end of combination treatment and after several weeks to more than one month later. At the end of SAHA exposure of spheroids irradiated with 6 Gy (FIG. 4, 4 days), the morphological appearance of the spheroids was similar to that observed for SAHA-only treated spheroids and is consistent with SAHA-induced apoptosis. In contrast, the morphology at 14 to 42 days shows cellular swelling and lysis, consistent with necrotic death.

The earliest evidence of a divergence in spheroid fate between SAHA-only and combination treatment is observed in the Ki67 staining (FIGS. 6A-C and 7B). Forty-eight hours after the end of incubation with 5 μM SAHA alone, proliferating cells were observed whereas no such restoration was seen in the spheroids treated with SAHA and radiation; at the dosage used, radiation alone did not alter cellular proliferation as evaluated by Ki67 staining (FIG. 6C, Panel H). Taken together, the proliferation and apoptosis data suggest that the enhanced effect of SAHA with radiation is due primarily to a decrease in subsequent proliferation of cells following incubation with SAHA rather than increased radiation-induced apoptosis.

The inability of cells to resume cycling after exposure to combination SAHA and radiation therapy can point to a disruption in repair of radiation induced damage or to enhancement of otherwise repairable DNA damage.

While this invention has been particularly shown and described with references to preferred embodiments thereof, it will be understood by those skilled in the art that various changes in form and details can be made therein without departing from the scope of the invention encompassed by the appended claims.

What is claimed is:

1. A method for treating cancer in a patient in need thereof comprising administering to said patient a first amount of a histone deacetylase inhibitor in a first treatment procedure, and a second amount of radiation in a second treatment procedure wherein, the first and second amounts together comprise a therapeutically effective amount.

2. The method according to claim 1, wherein said HDAC inhibitor is a hydroxamic acid derivative, a Short Chain Fatty Acid (SCFA), a cyclic tetrapeptide, a benzamide derivative, or an electrophilic ketone derivative.

3. The method according to claim 2, wherein said HDAC inhibitor is a hydroxamic acid derivative selected from the group consisting of SAHA, Pyroxamide, CBHA, Trichostatin A (TSA), Trichostatin C, Salicylihydroxamic Acid (SBHA), Azelaic Bishydroxamic Acid (ABHA), Azelaic-1-Hydroxamate-9-Anilide (AAHA), 6-(3-Chlorophenylureido) carpoic Hydroxamic Acid (3Cl-UCHA), Oxamflatin, A-161906, Scriptaid, PXD-101, LAQ-824, CHAP, MW2796, and MW2996.

4. The method according to claim 2, wherein said HDAC inhibitor is a Cyclic Tetrapeptide selected from the group consisting of Trapoxin A, FR901228 (FK 228 or Depsipeptide), FR225497, Apicidin, CHAP, HC-Toxin, WF27082, and Chlamydocin.

5. The method according to claim 2, wherein said HDAC inhibitor is a Short Chain Fatty Acid (SCFA) selected from the group consisting of Sodium Butyrate, Isovalerate, Valerate, 4 Phenylbutyrate (4-PBA), Phenylbutyrate (PB), Propionate, Butyramide, Isobutyramide, Phenylacetate, 3-Bromopropionate, Tributyrin, Valproic Acid and Valproate.

6. The method according to claim 2, wherein said HDAC inhibitor is a Benzamide derivative selected from the group consisting of CI-994, MS-27-275 (MS-275) and a 3′-amino derivative of MS-27-275.

7. The method according to claim 2, wherein said HDAC inhibitor is an electrophilic ketone derivative selected from the group consisting of a trifluoromethyl ketone and an α-keto amide.

8. The method according to claim 2, wherein said HDAC inhibitor is Depudecin.

9. The method according to claim 1, wherein said HDAC inhibitor is represented by the following structure: 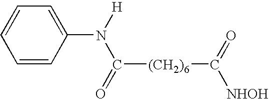
or a pharmaceutically acceptable salt thereof.

10. The method according to claim 1, wherein said HDAC inhibitor is pyroxamide, represented by the structure: 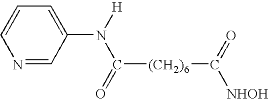
or a pharmaceutically acceptable salt thereof.

11. The method according to claim 1, wherein said HDAC inhibitor is represented by the structure: 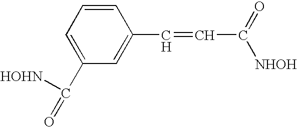
or a pharmaceutically acceptable salt thereof.

12. The method according to claim 1, wherein said HDAC inhibitor is represented by the structure: 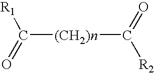
or pharmaceutically acceptable salts, solvates or hydrates thereof wherein:
R₁ and R₂ can be the same or different;
when R₁ and R₂ are the same, each is a substituted or unsubstituted arylamino cycloalkylamino or piperidino group;
when R₁ and R₂ are different R₁═R₃—N—R₄, wherein each of R₃ and R₄ are independently the same as or different from each other and are a hydrogen atom, a hydroxyl group, a substituted or unsubstituted, branched or unbranched alkyl, alkenyl, cycloalkyl, aryl, alkyloxy, aryloxy, arylalkyloxy group, or R₃ and R₄ are bonded together to form a piperidine group;
R₂ is a hydroxylamino, hydroxyl, amino, alkylamino, dialkylamino or alkyloxy group; and
n is an integer from about 4 to about 8.

13. The method according to claim 1, wherein said HDAC inhibitor is represented by the structure: 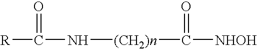
or pharmaceutically acceptable salts, solvates or hydrates thereof wherein:
R is a substituted or unsubstituted phenyl, piperidino, thiazolyl, 2-pyridyl, 3-pyridyl or 4-pyridyl group; and
n is an integer from about 4 to about 8.

14. The method according to claim 1, wherein said HDAC inhibitor is represented by the structure: 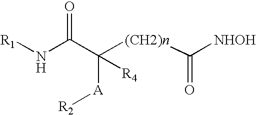
or pharmaceutically acceptable salts, solvates or hydrates thereof, wherein:
A is an amide moiety;
R₁ and R₂ are each selected from a substituted or unsubstituted aryl, arylamino, arylalkylamino, arylalkyl, aryloxy or arylalkyloxy group;
R₄ is hydrogen, a halogen, a phenyl or a cycloalkyl group; and
n is an integer from about 3 to about 10.

15. The method according to claim 1, wherein the radiation of the second treatment procedure is external beam radiation.

16. The method according to claim 1, wherein the radiation of the second treatment procedure is a radiopharmaceutical agent.

17. The method of claim 16, wherein the radiopharmaceutical is a radioactive conjugate.

18. The method according to claim 17, wherein said radioactive conjugate is a radiolabeled antibody.

19. The method according to claim 1, wherein the radiation is selected from the group consisting of: electromagnetic radiation and particulate radiation.

20. The method according to claim 19, wherein the electromagnetic radiation is selected from the group consisting of: x-rays, gamma rays and any combination thereof.

21. The method of claim 19, wherein the particulate radiation is selected from the group consisting of: electron beams (beta particles), protons beams, neutron beams, alpha particles and negative pi mesons.

22. The method of claim 21, wherein the particulate radiation is alpha particles.

23. The method according to claim 1, wherein a total of at least about 1 Gy of radiation is administered to the patient.

24. The method according to claim 1, wherein a total of at least about 10 Gy of radiation is administered to the patient.

25. The method according to claim 1, wherein a total of at least about 20 Gy of radiation is administered to the patient.

26. The method according to claim 1, wherein a total of at least about 40 Gy of radiation is administered to the patient.

27. The method according to claim 1, wherein the therapeutic effect of said HDAC inhibitor and said radiation is synergistic.

28. The method according to claim 26, wherein said HDAC inhibitor sensitizes cancer cells in the patient to radiation.

29. The method according to claim 1, wherein radiation sensitizes cancer cells in the patient to said HDAC inhibitor.

30. The method according to claim 1, wherein said UDAC inhibitor and radiation are administered simultaneously.

31. The method according to claim 1, wherein said HDAC inhibitor and said radiation are administered sequentially.

32. The method according to claim 31, wherein said HDAC inhibitor is administered prior to administering said radiation.

33. The method according to claim 31, wherein said HDAC inhibitor is administered after administering said radiation.

34. The method of claim 1, wherein the HDAC inhibitor is administered orally, parenterally, intraperitoneally, intravenously, intraarterially, transdermally, sublingually, intramuscularly, rectally, transbuccally, intranasally, via inhalation, vaginally, intraoccularly, locally, subcutaneously, intraadiposally, intraarticularly, intrathecally.

35. The method of claim 1, wherein the HDAC inhibitor is in a slow release dosage form.

36. The method of claim 16, wherein the radiopharmaceutical agent is administered orally, parenterally, intraperitoneally, intravenously, intraarterially, transdermally, sublingually, intramuscularly, rectally, transbuccally, intranasally, via inhalation, vaginally, intraoccularly, locally, subcutaneously, intraadiposally, intraarticularly or intrathecally.

37. The method of claim 16, wherein the radiopharmaceutical agent is in a slow release dosage form.

38. A method of determining the sensitivity of a cancer cell to a combination therapy of an HDAC inhibitor and radiation, said method comprising the step of contacting said cancer cell with a first amount of a histone deacetylase inhibitor in a first treatment procedure, and a second amount of radiation in a second treatment procedure, wherein the first and second treatments together comprise a therapeutically effective amount and assessing the sensitivity of the cell to treatment.

39. A method of determining a therapeutically effective amount of a combination of an HDAC inhibitor and radiation for treating a cancer, comprising the step of exposing a cancer cell to a first amount of a histone deacetylase inhibitor in a first treatment procedure, and a second amount or dose of radiation in a second treatment procedure, wherein the first and second treatments together comprise a therapeutically effective amount and assessing the anticancer effects.

40. A pharmaceutical composition comprising a first amount of a histone deacetylase inhibitor and a second amount of radiation wherein the first and second amounts together comprise a therapeutically effective amount.

41. The composition of claim 40, wherein the radiation is a radiopharmaceutical agent.

42. Use of a first amount of an HDAC inhibitor and a second amount of radiation for the manufacture of a medicament for treating cancer.

43. The use of claim 42, wherein the radiation is a radiopharmaceutical agent.
